(19) United States
(12) Patent Application Publication
(10) Pub. No.: US 2019/0070396 A1
(43) Pub. Date: Mar. 7, 2019

(54) APPARATUS AND METHODS FOR RAPID TRANSMUCOSAL DRUG DELIVERY

(71) Applicant: Statim Pharmaceuticals, Inc., Half Moon Bay, CA (US)

(72) Inventors: Randolph M. Johnson, Half Moon Bay, CA (US); Nooshin T. Azimi, Menlo Park, CA (US); Edward F. Schnipper, Redwood City, CA (US); Thomas H. Cauley, III, Redwood City, CA (US)

(73) Assignee: Statim Pharmaceuticals, Inc., Half Moon Bay, CA (US)

(21) Appl. No.: 16/182,395

(22) Filed: Nov. 6, 2018

Related U.S. Application Data
(63) Continuation of application No. PCT/US2018/039449, filed on Jun. 26, 2018.
(60) Provisional application No. 62/526,251, filed on Jun. 28, 2017.

(51) Int. Cl. A61M 31/00; A61K 31/137; A61K 31/485; A61K 9/00; A61K 47/08; A61K 47/44; A61M 37/00

(52) U.S. Cl. CPC A61M 31/00; A61K 31/137; A61K 31/485; A61K 9/006; A61M 2207/00; A61K 47/44; A61M 37/00; A61M 2210/0625; A61K 47/08

(57) ABSTRACT

Medical devices and methods for rapid and efficient systemic drug delivery via mucous membranes, particularly oral mucosae, are described. Use of such devices and methods are particularly easy for people without any medical training to employ should they be called upon to provide emergency medical treatment for a victim suffering from anaphylactic shock, opioid overdose, or other life-threatening events. In some embodiments, the device includes an applicator tip with a porous application layer positioned on the end of an elongate handle. The device may also include various means for disrupting a barrier to facilitate mixing of compounds, including an active ingredient, and permit flow of the compounds from a reservoir to the applicator tip.

### CROSS-REFERENCE TO RELATED APPLICATIONS

This application is a continuation of PCT Patent Application No. PCT/US2018/039449, filed on Jun. 26, 2018, entitled “Apparatus and Methods for Rapid Transbuccal Drug Delivery,” which claims priority to U.S. Provisional Patent Application No. 62/526,251, filed Jun. 28, 2017, entitled “Apparatus and Methods for Rapid Transbuccal Drug Delivery,” the disclosures of which are hereby incorporated by reference in their entirety for all purposes.

### BACKGROUND OF THE INVENTION

Treating medical emergencies can be extremely challenging for even the most experienced and best trained health care professionals. Current therapy for anaphylaxis or opioid overdose involves the use of antidote medication provided via intramuscular injection (e.g., EpiPen® or Evzio®, respectively) or intranasal administration (e.g., Adrenalin® Chloride Solution or Narcan®, respectively). Intranasal application can prove virtually useless in the presence of nasal congestion, mucous discharge, blood, or vasoconstrictors (e.g., amphetamines, antihistamines, cocaine), for example. These injectable and nasal routes have proven difficult (if not impossible) for untrained persons to employ because they are complicated, messy and unfamiliar. They are also potentially dangerous due to risk of needle sticks, accidental misfiring, exposure to blood-borne pathogens, infection, and incorrect dosage applications, for example. Fear, discomfort and apprehension leads to under-utilized administration of these life-saving drugs. These devices can also be expensive. Kaléo, Inc., the manufacturer of Evzio®, charges nearly $4500 for a two-pack of auto injectors and Adapt Pharma, Inc., the maker of Narcan®, charges about $150 for a two-pack of nasal spray.

Anaphylaxis is a severe, potentially fatal allergic reaction. Food allergy is the most common cause of anaphylaxis, although several other allergens (e.g., insect stings, medications, or latex) are also potential triggers. At least 1.6 percent of Americans, and possibly as many as 5.1 percent, have experienced anaphylaxis. (Wood, R. A., et al., Anaphylaxis in America: the prevalence and characteristics of anaphylaxis in the United States, J. Allergy Clin. Immunol. (2014) 133(2):461-467). It is imperative to quickly treat anaphylactic shock to avoid complications including death. However, it is estimated that 45% of parents are uncomfortable using the EpiPen® (Kim, et al., Parental use of EpiPen for children with food allergies, J. Allergy Clin. Immunol. (2005) 116:164-168) and less than 30% of patients carry adrenaline auto-injectors (Song, et al., Anaphylaxis treatment: current barriers to adrenaline auto-injector use, Allergy (2014) 69:983-991).

Opioids is a class of drugs that includes the illegal drug heroin and synthetic opioids such as fentanyl and methadone. Examples of pain relievers available legally by prescription include fentanyl, oxycodone (e.g., OxyContin®), hydrocodone (e.g., Vicodin®), codeine, morphine, and many others. On average, 122 people die every day from overdoses of prescription and illegal opioids, making the opioid crisis the worst addiction epidemic in U.S. history (Time, Mar. 5, 2018). Total deaths linked to drug overdoses have roughly quadrupled over the past two decades, with the surge blamed largely on opioids. About 64,000 fatal overdoses occurred in 2016, and 42,000 of those deaths were linked to opioids, according to the Centers for Disease Control and Prevention. The alarming trend of “stretching” heroin to create a “monster” dose by adding carfentanil will surely increase fatal overdoses (a.k.a. “grey death”) unless treatment can be expedited, because carfentanil is 5,000 times more potent than a unit of heroine. Furthermore, each year opioid abuse imposes about $55 billion in health and social costs and imposes approximately $20 billion in costs for emergency and inpatient care across the United States (Law360, May 2, 2018.) By one estimate, private insurance claims related to opioid dependence rose by 3,200% nationally from 2007 to 2014 (Law360, May 2, 2018). To address this growing problem, the wider availability of overdose-reversal drug naloxone has been proposed, among other initiatives.

There is clearly an unmet medical need to have more user-friendly, needle-free, delivery systems available for medical professionals, emergency first responders, family members, friends, and even the general public to use to treat anaphylaxis or opioid overdose. By making easy-to-use devices available to administer epinephrine or naloxone to victims, laypersons and emergency medical responders will be more confident and less apprehensive to first manage these types of emergencies. Development and approval of reliable non-injectable formulations will facilitate wider therapeutic drug provision across communities.

Since the treatment need is urgent and the availability of trained persons is not always present, there is an acute need for a treatment method that is simple and effective and can be administered easily without training. An inexpensive treatment with drug stability at ambient temperature and widespread availability to laypersons would also be welcome to increase the odds of patient survival.

Information related to attempts to address these problems can be found in U.S. Pat. Nos. 3,759,375; 6,899,897; 6,959,808; 8,648,082; 8,709,439; and 8,846,092; and United States Patent Application Publication Numbers: 2004/0191274 A1; 2008/0286299 A1; 2008/0317690 A2; 2009/0004252 A1; 2010/0247586 A1; 2011/0281771 A1; 2012/0220578 A1; 2013/01748463 A1; 2014/0371210 A1; 2015/0051200 A1; 2015/0250887 A1; and 2015/0306362 A1 as well as European Patent Number EP 310876 A1, and International Patent Publication Numbers WO 01/74321 A2, WO 01/89476 A1, WO 2005/065640 A1, WO 2005/009386 A2, WO 2005/009396 A2, WO 2006/124366 A2, and WO 2008/156559 A2; and the following journal articles and publications: Advisory Committee of Oct. 5, 2016, Naloxone for treatment of opioid overdose, Insys Development Company, Inc., pp. 1-11; Dart, R. C., et al., Trends in opioid analgesic abuse and mortality in the United States, N. Engl. J. Med. (2015); 372(3):241-248; de Vries, M. E., et al., Developments in buccal drug delivery, Critical Reviews in Therapeutic Drug Carrier Systems (1991) 8(3):271-303; Ezhumalai, et al., Medicated Chewing Gum—A Novel Drug Delivery Technique for Systematic and Targeted Drug Delivery Introduction, International Journal of Pharmacy & Technology (2011); 725-744; Kim, J. S., et al., Parental use of EpiPen® for children with food allergies, J. Allergy Clin. Immunol. (2005); 116:164-168; Krieter, P., et al., Pharmacokinetic properties and human use characteristics of an FDA-approved intranasal naloxone product for the treatment of opioid overdose, J. Clin. Pharmacol. (2016); 56(10) 1243-1253; Madhav, N. V. S., et al., Orotransmucosal drug delivery systems: A review, J. Controlled Release (2009); 140:2-11; National Institutes on Drug Abuse (NIDA), Prescription opioids and heroin, January 2018, pages 1-19, www.drubabuse.gov; National Institutes on Drug Abuse (NIDA), Naloxone for opioid overdose: Life-Saving Science, March 2017, 2 pages total, www.drubabuse.gov; National Institutes on Drug Abuse (NIDA), Effective treatments for opioid addition, November 2016, 2 pages total, www.drubabuse.gov; Patel, V. F., et al., Advances in oral transmucosal drug delivery, J. Controlled Release (2011); 153(2):106-116; Sanz, R., et al., Enhancing topical analgesic administration: review and prospect for transdermal and transbuccal drug delivery systems, Curr. Pharm. Des. (2015) 21(20):2867-82; Shojaei, A. H., et al., Buccal mucosa as a route for systemic drug delivery: A review, J. Pharm. Pharmacet. Sci. (1998); 1(1):15-30; Song, T. T., et al., Anaphylaxis treatment: Current barriers to adrenaline auto-injector use, Allergy (2014); 69:983-991; Wood, J. P., et al., Safety of epinephrine for anaphylaxis in emergency setting, World J. Emerg. Med. (2013); 4(4): 245-251; and Wood, R. A., et al., Anaphylaxis in America: the prevalence and characteristics of anaphylaxis in the United States, J. Allergy Clin. Immunol. (2014) 133(2):461-467, for example.

Various types of methods and devices for rapid transmucosal drug delivery, including some embodiments of the invention, can mitigate or reduce the effect of, or even take advantage of, some or all of these potential problems.

For the foregoing reasons, there is a legitimate and long-felt need for simple, inexpensive, effective and efficient ways for an untrained person to provide prompt drug administration via mucosal tissues, particularly the oral mucosa, during medical emergencies.

### BRIEF SUMMARY OF THE INVENTION

The present invention provides devices and related methods of use and manufacture for delivery of a pharmaceutically active ingredient. Such devices can be used for the delivery of any active ingredient. As disclosed herein, such devices are particularly useful in emergency situations for delivery of pharmaceutical formulations including, for example, naloxone or epinephrine, as discussed in greater detail below.

According to one embodiment of the invention, a hand-held device for the urgent delivery of a pharmaceutically active ingredient during a medical emergency comprises: (a) an elongated rigid or semi-rigid handle having an end, (b) an applicator tip, the applicator tip comprising: (i) a drug reservoir configured to contain a pharmaceutical composition comprising an effective amount of the active ingredient (e.g., epinephrine or naloxone), wherein the drug reservoir comprises an exterior surface that is formed in or attached to the end of the handle, and an open end; (ii) a porous application layer (e.g., an absorbent foam) comprising an exterior surface configured to spread the pharmaceutical composition at a delivery site and an interior surface; (iii) a barrier between the open end of the drug reservoir and the interior surface of the application layer and configured to prevent flow of the pharmaceutical composition from the drug reservoir to the application layer. The barrier is further configured to rupture when sufficient pressure is applied to the barrier to permit flow of the pharmaceutical composition from the drug reservoir into the application layer; and (c) a removable protective covering over the exterior surface of the application layer or the applicator tip. Optionally, the application layer is configured to abrade the oral tissue at the delivery site. The pharmaceutical composition may comprise a unit dose of the active ingredient. In one such embodiment, the pharmaceutical composition comprises the pharmaceutically active ingredient, a resin, a volatile solvent, and optionally water.

In one such device, the barrier comprises a tab that is configured to displace or disrupt the barrier when the tab is pulled. Alternatively, the barrier may comprise a button attached to the interior surface of the application layer, the button comprising one or more spiky projections that extend toward the nonporous barrier. The button is configured to pierce the barrier to permit flow of the pharmaceutical composition from the drug reservoir to the application layer when pressure is exerted against the exterior surface of the application layer.

According to another embodiment of the invention, devices are provided for urgent delivery of a pharmaceutically active ingredient (e.g., epinephrine or naloxone). Such devices comprise: (a) an elongated rigid or semi-rigid handle having an end; (b) an applicator tip comprising: (i) a rigid, non-porous backing comprising an exterior surface that is formed in or attached to the end of the handle and an interior surface; and (ii) a porous application layer comprising a first surface in contact with the interior surface of the backing and a second surface configured to spread the pharmaceutical composition on the oral mucosa of a patient. The application layer contains a pharmaceutical composition comprising an effective amount of the active ingredient (e.g., a unit dose); and (c) a removable protective covering surrounding the applicator tip. In such a device, the application layer comprises an absorbent foam, a hook-and-eye fastener material, or nylon bristles, for example. The exterior surface of the application layer may optionally be configured to abrade the oral mucosa at a drug delivery site. The drug delivery site is buccal, sublingual or labial. The oral mucosa may be buccal mucosa and the active ingredient may be an adrenergic hormone or a derivative of morphine. In one such embodiment, the pharmaceutical composition comprises the pharmaceutically active ingredient, a resin, a volatile solvent, and optionally water.

According to another embodiment of the invention, a method of delivering a pharmaceutically active ingredient to a mouth of a patient requiring emergency medical care comprises providing a device having an elongated handle at a first end and an applicator tip at a second end. The applicator tip contains a pharmaceutically active ingredient The first end of the elongate handle of the device is grasped by a hand of a user and the applicator tip is positioned substantially in contact with an oral tissue in the mouth of the patient. The exterior surface of an application layer is configured to abrade the oral tissue. Optionally, the applicator tip is rubbed against the oral tissue in the mouth of the patient. The oral tissue includes mucosae selected from the group consisting of buccal mucosa, sublingual mucosa and labial mucosa. The oral tissue may be buccal mucosa.

According to yet another embodiment of the invention, a hand-held device for urgent systemic delivery of a pharmaceutically active ingredient and a physiologically acceptable carrier across oral mucosae comprises an applicator tip. The tip comprises a porous application layer comprising an exterior surface configured to receive and apply the pharmaceutically active ingredient to the oral mucosa. The device also comprises an elongated rigid or semi-rigid handle having an end. The handle comprises: (i) a first internal reservoir configured to contain an effective amount of the active ingredient; (ii) a second internal reservoir configured to contain the physiologically acceptable carrier; (iii) a barrier between the first and second reservoirs configured to prevent mixing of the active ingredient with the physiologically acceptable carrier; and (iv) a means for moving or disrupting the barrier to permit mixing of the active ingredient and the physiologically acceptable carrier. The mixture of the active ingredient and the carrier flows from the handle into the application layer of the application tip. The device also comprises a removable protective covering over the exterior surface of the application layer or the applicator tip.

The active ingredient is naloxone or epinephrine. The physiologically acceptable carrier is a diluent and the diluent is ethanol. The means for moving or disrupting the barrier comprises a partition that is configured to displace or disrupt the barrier when the handle is twisted and/or bent. The application layer contains a plant-based resinous gum configured to provide mucosadhesive properties. The aforementioned oral mucosae is the buccal mucosa.

According to another embodiment of the invention, a hand-held device for urgent systemic delivery of a pharmaceutical composition across oral mucosae comprises an applicator tip comprising a porous application layer comprising an exterior surface configured to receive and apply the pharmaceutical composition to the oral mucosae. The device also comprises an elongated rigid or semi-rigid handle having an end. The handle comprises: (i) an internal reservoir configured to contain an effective amount of the pharmaceutical composition, the composition includes a pharmaceutically active ingredient and a physiologically acceptable carrier; (ii) a barrier between the reservoir and applicator layer configured to prevent the pharmaceutical composition from flowing to the application layer of the application tip; and (iii) a means for moving or disrupting the barrier to permit flow of the pharmaceutical composition from the internal reservoir in the handle to the application layer of the application tip.

The active ingredient is naloxone or epinephrine and the physiologically acceptable carrier is ethanol. The application layer contains a plant-based resin and the plant-based resin is benzoin gum or badam gum. The device further comprises a removable protective covering over the exterior surface of the application layer or the applicator tip. The barrier between the reservoir and applicator layer is a foil blister, the applicator tip is a flocked pad material and the means for moving or disrupting the barrier to permit flow of the pharmaceutical composition from the internal reservoir in the handle to the application layer of the application tip includes breaking the foil blister.

In yet another embodiment of the invention, a hand-held device for urgent systemic delivery of a pharmaceutical composition across oral mucosae. The device comprises an applicator tip, a handle, and a housing. The applicator tip comprises an exterior surface configured to receive and apply the pharmaceutical composition to the oral mucosae. The elongated rigid or semi-rigid handle has a grasping end. The applicator tip is positioned on the handle opposite the grasping end. The removable housing is configured to enclose the applicator tip and a portion of the elongate handle excluding the grasping end. The removable housing contains: (i) a slidable shuttle having a first side, a second side, and at least one opening between the sides; (ii) an internal reservoir configured to contain an effective amount of the pharmaceutical composition. The composition includes a pharmaceutically active ingredient and a physiologically acceptable carrier. The reservoir is positioned on the first side of the slidable shuttle and positioned in communication with the at least one opening. The applicator tip is positioned on the second side of the slidable shuttle and positioned in communication the at least one opening; (iii) a barrier between the reservoir and applicator tip prevents the pharmaceutical composition from flowing from the reservoir through the at least one opening to the applicator tip; and (iv) a means for moving the applicator tip and the shuttle to disrupt the barrier so as to permit flow of the pharmaceutical composition from the internal reservoir through the at least one opening onto the exterior surface of the applicator tip.

The means for moving the applicator tip and the shuttle to disrupt the barrier includes holding the removable housing while pulling the grasping end of the handle away from the housing until the application tip is separated from the housing. The active ingredient is naloxone or epinephrine. The physiologically acceptable carrier is ethanol and the application layer contains a plant-based resin. The plant-based resin is benzoin gum or badam gum. The reservoir is a blister containing between about 100 μL and 500 μL of the pharmaceutical composition. The barrier is disrupted when the reservoir contacts a fixed portion of the housing.

In another embodiment of the invention, a hand-held device to expedite delivery of a pharmaceutically active ingredient to an oral cavity of a patient comprises an elongated rigid or semi-rigid handle having an end. The handle comprises a first reservoir configured to contain a diluent. The first reservoir comprises an exterior surface that is formed in or attached to the end of the handle, and an open end. The device also comprises an applicator tip comprising: (i) a second reservoir configured to contain an effective amount of the active ingredient, wherein the second reservoir comprises an exterior surface that is attached to the end of the handle, and an open end; (ii) a porous application layer comprising an exterior surface configured to receive and spread the diluent and active ingredient to the oral cavity of the patient when mixed together; (iii) a barrier between the first and second reservoirs configured to prevent mixing of the diluent and the active ingredient; and (iv) a means for moving or disrupting the barrier to permit flow of the diluent to the applicator tip and mixing with the active ingredient in the application layer. The device also comprises a removable protective covering over the exterior surface of the application layer or the applicator tip.

The means for moving or disrupting the barrier comprises moving the applicator tip in a direction while the handle remains substantially stationary to displace or disrupt the barrier when movement is applied. Moving the applicator tip includes twisting, bending, pulling, compressing, or any combination thereof. The application layer is configured to spread the diluent and active ingredient substantially across buccal mucosa.

In yet another embodiment of the invention, a method of delivering a pharmaceutically active ingredient to a mouth of a patient requiring emergency medical care from a person comprises providing the person with a device. The device has an elongated handle positioned between a grasping end and an applicator tip. The applicator tip is configured to be removably contained within a housing. The housing contains a reservoir filled with the pharmaceutically active ingredient as well as a mechanism for breaking the reservoir. The housing is held with a first hand of the person. The person uses their second hand to pull the grasping end of the device to remove the applicator tip of the device from the housing. Pulling the applicator tip causes the reservoir to break and flow the pharmaceutically active ingredient onto the applicator tip as the tip is being pulled apart from the housing. The housing can now be discarded. An opening is created (by the person) to insert the device in the mouth of the patient by pulling the patient's cheek away from their mouth. The applicator tip is inserted into the opening and substantially positioned in contact with an oral tissue of the patient. Optionally, the applicator tip can be rubbed against the oral tissue of the patient.

In another embodiment of the invention, a method of manufacturing a hand-held device for urgent delivery of a pharmaceutically active ingredient to a mucosa of a patient comprises providing and molding a medical grade material for manufacturing an elongated rigid or semi-rigid handle. The handle has an end. A medical grade material for manufacturing an applicator tip is also provided and molded. The applicator tip comprises a drug reservoir configured to contain a pharmaceutical composition comprising an effective amount of the active ingredient. The drug reservoir comprises an exterior surface that is formed in or attached to the end of the handle and an open end. A porous application layer comprises an exterior surface configured to spread the pharmaceutical composition at a delivery site and an interior surface. A barrier between the open end of the drug reservoir and the interior surface of the application layer is configured to prevent flow of the pharmaceutical composition from the drug reservoir to the application layer. The applicator tip also comprises a means for moving or disrupting the barrier to permit flow of the pharmaceutical composition from the drug reservoir into the application layer and a removable protective covering over the exterior surface of the application layer or the applicator tip. The pharmaceutical composition is placed in the drug reservoir and sealed. The hand-held device is assembled and packaged.

In another embodiment of the invention, a method of treating an individual comprises providing a device. The device comprises: i) an elongated rigid or semi-rigid handle having an end; ii) an applicator tip formed in or attached to the end of the handle; and iii) a removable protective covering over the exterior surface of the application layer or the applicator tip. The applicator tip comprises a drug reservoir configured to contain a pharmaceutical composition comprising an effective amount of naloxone or epinephrine. The drug reservoir comprises an exterior surface that is formed in or attached to the end of the handle, and an open end; and an exterior surface in fluid communication with the drug reservoir that is configured to spread the pharmaceutical composition on an oral mucosa of the individual. The method of treating an individual also comprises removing the protective covering, inserting the tip into the mouth of the individual; and spreading the pharmaceutical composition on the oral mucosa. The individual may be unconscious or unresponsive.

In yet another embodiment of the invention, a kit for the urgent delivery of a pharmaceutically active ingredient to a mucosa of a patient during a medical emergency comprises a device of any of the preceding claims, instructions for using the device, and a package containing the device and the instructions for use.

These and other features, aspects, and advantages of various embodiments of the invention will become better understood with regard to the following description, appended claims, accompanying drawings and abstract.

### BRIEF DESCRIPTION OF THE DRAWINGS

FIG. 1A is a cut-a-way perspective view of the buccal mucosa.

FIG. 1B is a cross-sectional diagram of tissue layers found in the oral mucosa.

FIG. 2 shows a device, according to some embodiments of the invention, in which a pharmaceutical formulation containing an active ingredient is contained within the applicator tip.

FIG. 3 shows a two-compartment device, according to some embodiments of the invention, in which the drug reservoir and applicator tip are separate, isolated compartments.

FIG. 4 shows an alternative two-compartment device according to some embodiments of the invention.

FIGS. 5A-5B show a two-compartment device in which the handle includes two separate, isolated reservoirs according to some embodiments of the invention.

FIG. 6A shows a device in which the handle includes a separate, isolated reservoir according to some embodiments of the invention.

FIG. 6B shows a device in which the handle includes a separate, isolated reservoir according to other embodiments of the invention.

FIG. 7 shows a device in which the handle includes a separate, isolated reservoir and the applicator includes a drug reservoir according to some embodiments of the invention.

FIG. 8A shows a perspective view of a device with a separate reservoir in a housing according to some embodiments of the invention.

FIG. 8B shows a cross-sectional view of the device of FIG. 8A.

FIG. 8C shows a cross-sectional view of the device and housing according to some embodiments of the invention.

FIG. 8D shows a cross-sectional perspective view of the device with the top portion of the housing removed.

FIG. 8E depicts an exploded view including parts of the device, housing and assembly.

FIG. 9 illustrates the systemic delivery of selected transbuccally-administered naloxone concentrations over time according to some embodiments of the invention.

FIG. 10 shows a histogram of Tₘₐₓ comparisons of buccal versus intramuscular injection of naloxone.

FIG. 11 shows a histogram of Cₘₐₓ comparisons of buccal versus intramuscular injection of naloxone.

FIG. 12 shows a histogram of AUC comparisons of buccal versus intramuscular injection of naloxone.

FIG. 13 shows a table of pharmacokinetic parameters of buccal versus intramuscular injection versus intranasal spray application of naloxone.

FIG. 14 shows a histogram of Cₘₐₓ comparisons of buccal versus intramuscular injection of epinephrine.

FIG. 15 shows a flowchart illustrating a method of using the device to deliver a pharmaceutically active ingredient according to some embodiments of the invention.

FIG. 16 shows a flowchart illustrating another method of using the device to deliver a pharmaceutically active ingredient according to other embodiments of the invention.

FIG. 17 shows a kit according to some embodiments of the invention.

Other features of the present embodiments will be apparent from the accompanying drawings and from the detailed descriptive information that follows.

### DETAILED DESCRIPTION OF THE INVENTION

A medical emergency is a situation where response time is of the essence to save a patient who is usually unconscious, hypoxic, and in the more severe cases, apneic. The patient may be a human or lower animal (e.g., police canine or drug detection service dog). Initiating treatment as early as possible, even before the arrival of emergency medical services (EMS) at the scene, is a medical imperative and a critical determinant of outcome in opioid overdose and anaphylaxis. Expanding access to those individuals who are in frequent contact with a person or service animal at risk of overdose or shock, such as family members, friends, or peace officers, is also critical.

The term “buccal” and “oral composition” as used herein and in the appended claims denotes administering an active therapeutic agent/ingredient into the oral cavity of a subject/patient. The term “therapeutic agent,” as used herein and in the appended claims denotes a compound, including a protein or a peptide, that has active therapeutic, pharmacokinetic properties and utility. Illustrative categories of therapeutic agents suitable for practicing the present invention are anesthetics, antihistamines, antipsychotics, acetylcholinesterase inhibitors, analgesics, benzodiazepines, antipyretics, anticonvulsants, triptans/serotonin agonists, non-steroidal anti-inflammatory drugs (NSAIDS), antiemetics, corticosteroids, DDC inhibitors, proton pump inhibitors, antidepressants, anticholinergics, monoamine oxidase inhibitors (MAOIs), dopamine receptor antagonists, nonbenzodiazepine hypnotics, narcotics, nicotine replacement therapy agents, hormones, oral fungicides, opioid analgesics, small molecule therapeutics, vasodilators, vasoconstrictors, and the like.

As used herein, the term “physiologically acceptable carrier” refers to a diluent (i.e., a substance used to dilute something), adjuvant, excipient, or the like vehicle in which a therapeutic agent is administered. Such carriers can include alcohol, starch, glucose, lactose, sucrose, gelatin, malt, rice, flour, chalk, silica gel, sodium stearate, glycerol monostearate, talc, sodium chloride, dried skim milk, or any compound found in the Handbook of Pharmaceutical Excipients (4^{th} edition, Pharmaceutical Press) and the like. A minor amount of wetting or emulsifying agents, or pH buffering agents such as acetates, citrates, or phosphates may also be present. Also, antibacterial agents such as methyl parabens; antioxidants such as ascorbic acid or sodium metabisulfite; chelating agents such as ethylenediaminetetraacetic acid (EDTA); and agents for the adjustment of tonicity such as sodium chloride or dextrose may be present. Preservatives commonly known to those of skill in the art may also be present.

The term “therapeutically effective amount” refers to those amounts that, when administered to a particular subject in view of the nature and severity of that subject's disease or condition, will have a desired therapeutic effect, (e.g., an amount which will cure, prevent, inhibit, or at least partially arrest or partially prevent a target disease or condition).

Additionally, the term “Cₘₐₓ” refers to the maximum (i.e., peak) serum concentration that a drug achieves in a specified test area of the body after the drug has been administrated and before the administration of a second dose. Cₘₐₓ is the opposite of Cₘᵢₙ, which is the minimum (i.e., trough) concentration that a drug achieves after dosing. The related pharmacokinetic parameter Tₘₐₓ is the time at which the Cₘₐₓ is observed. The “area under the curve” or AUC ranges from zero to infinity and represents the total drug exposure over time. Assuming linear pharmacodynamics with elimination rate constant K, the AUC is proportional to the total amount of drug absorbed by the body.

A mucous membrane or mucosa is a membrane that lines various cavities in the body and covers the surface of internal organs. It consists of one or more layers of epithelial cells overlying a layer of loose connective tissue. Mucous membranes line many tracts and strictures of the body, including the mouth, nose, eyelids, trachea, and lungs, stomach and intestines, and the ureters, urethra, and urinary bladder.

Transporting drugs across mucosae of the oral cavity into the systemic circulation is an excellent alternative to injectable, aerosolized or ingested drugs—particularly during medical emergencies. Advantages include: 1) delivering the drug quickly; 2) bypassing hepatic metabolism and degradation in the digestive system; 2) localizing precise dosage; 3) allowing for unidirectional delivery (i.e., only oral mucosal absorption); and 4) using a delivery device that prevents diffusion-limiting mucus buildup. Transbuccal drug administration, in particular, is advantageous because the surface area of the buccal mucosa is relatively large, has good blood flow (i.e., vascularization), has high permeability with virtually no lipid layer, has a uniform temperature and the drug is not prone to salivary dilution.

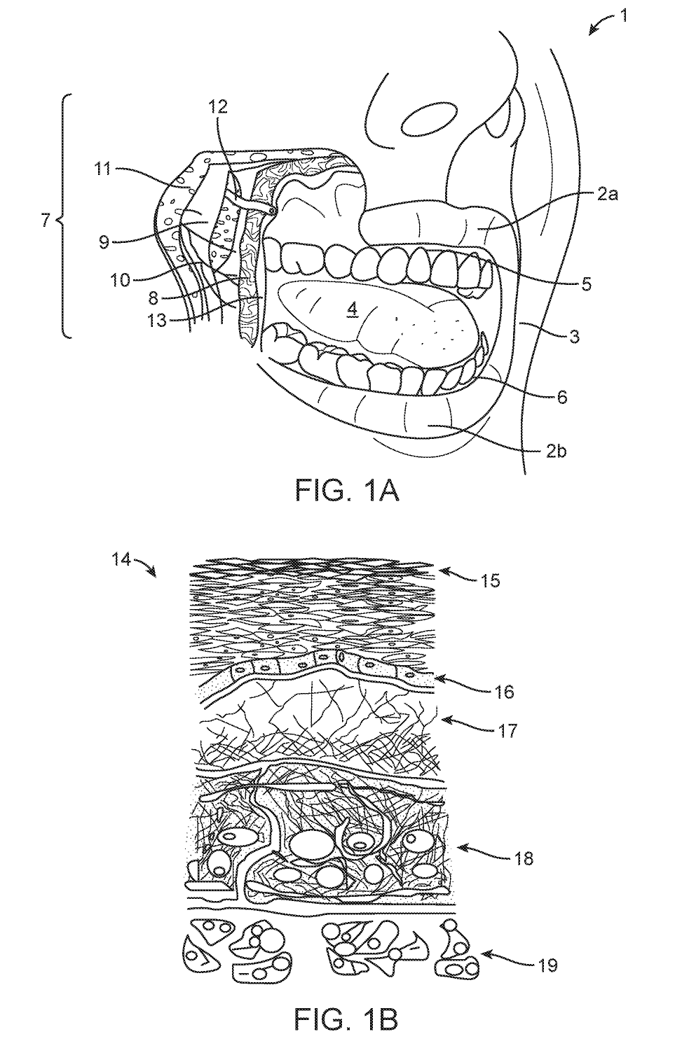

Referring to FIG. 1A, the mammalian oral cavity 1 comprises the lips 2a, 2b, cheek 3, tongue 4, hard palate, soft palate and floor of the mouth. The lining of the oral cavity is referred to as the oral mucosa, and includes the buccal mucosa 13, sublingual mucosa (located under the tongue), gingival mucosa 5, palatal mucosa and labial mucosa 6. In addition to the buccal mucosa 13, the buccal region 7 includes the buccinator muscle 8, masseter muscle 9, facial nerves 10, skin 11, and parotid duct 12. Three types of oral mucosa can be found in the oral cavity; the lining mucosa is found in the outer oral vestibule (the buccal mucosa 13) and the sublingual region (floor of the mouth under the tongue). The buccal, sublingual, and the mucosal tissues at the ventral surface of the tongue account for about 60% of the total oral mucosal surface area.

As shown in FIG. 1B, the top quarter to one-third of the oral mucosa 14 is made up of closely compacted epithelial cells 15 followed by the basement membrane 16 and lamina propia 17. The sub-mucosa contains blood vessels 18 and nerves followed by a muscle or bone layer 19 located most internal. A comparison of different regions in the oral cavity are shown in the table below (Patel, V. F., et al., Advances in oral transmucosal drug delivery, J. Controlled Release 2011; 153(2): 106-116).

| Tissue | | Thickness | Turnover | Surface Area | | Residence | Blood |
|---|---|---|---|---|---|---|---|
| Location | Structure | (μm) | Time (days) | (cm² ± SD) | Permeability | Time | Flow* |
| Buccal | NK | 500-600 | 5-7 | 50.2 ± 2.9 | Intermediate | Intermediate | 20.3 |
| Sublingual | NK | 100-200 | 20 | 26.5 ± 4.2 | Very Good | Poor | 12.2 |
| Gingival | K | 200 | — | — | Poor | Intermediate | 19.5 |
| Palatal | K | 250 | 24 | 20.1 ± 1.9 | Poor | Very Good | 7.0 |

NK is nonkeratinized tissue,
K is Keratinized tissue and
*in rhesus monkeys (ml/min/100 g tissue).

Due to its unique structural and physiological properties, the oral mucosa 14 offers several opportunities for systemic drug delivery. As the mucosa is highly vascularized, any drug diffusing across the oral mucosa membranes has direct access to the systemic circulation via blood vessels 18 and venous drainage and will bypass hepatic metabolism altogether. The rate of blood flow through the oral mucosa 14 is substantial, and is generally not considered to be the rate limiting factor in the absorption of drugs by this route. Enzyme degradation in the gastrointestinal (GI) tract is a major concern for oral drug delivery. In comparison, the buccal and sublingual regions have fewer enzymes and lower enzyme activity, which is especially favorable to protein and peptide delivery. The enzymes present in buccal mucosa include aminopeptidases, carboxypeptidases, dehydrogenases and esterases, for example. The buccal and sublingual routes are preferred for drug delivery via the oral mucosa because of the higher overall permeability compared to the other mucosa of the mouth. Drugs can be transported across epithelial membranes 15 by passive diffusion, carrier-mediated active transport or other specialized mechanisms. The predominant mechanism of buccal absorption is passive diffusion across lipid membranes via paracellular and/or transcellular pathways. While not wanting to be bound by any particular theory, it is believed that drugs may achieve immediate and intracellular loading of the active ingredient into the bloodstream by way of the jugular vein.

Illustrative opioid receptor antagonists suitable for buccal administration and absorption are naloxone, naltrexone and nalmefene. Illustrative opioid analgesics (i.e., narcotics) are morphine and morphine derivatives such as fentanyl, buprenorphine, carfentanil, and sulfentanil. Example non-steroidal anti-inflammatory agents (NSAIDs) include acylpropionic acid derivatives, such as ibuprofen, salicylic acid derivatives, and the like. Example anticonvulsants include iamotrigine, phenobarbital, phenytoin, and the like. Example benzodiazepines include clonazepam, diltiazem, particularly diltiazem hydrochloride (DHCl), and the like. Example triptans/serotonin agonist includes rizatriptan, zolmitriptan, and the like. Example antiemetics include ondansetron, ondansetron hydrochloride (ODAN.HCl), scopolamine, and the like. Example local anesthetics include lidocaine, particularly lidocaine hydrochloride (LHCl). Example nicotine replacement therapy agents include nicotine hydrogen tartrate (NHT).

Some more examples of drugs and other active ingredients transported through the buccal mucosa include, but are not limited to, epinephrine, flecainide, naltrexone, buprenorphine, nalbuphine, alphaprodine, pethidine, lignocaine, codeine, febuverin, cetylpyridinium chloride, tetracylcline, metronidazole, sotalol, lamotrigine, galantamine, buspirone, glyceryl trinitrate, isosorbide dinitrate, monocarboxylic acids, glucose, asenapine, nitroglycerin, captopril, nifedipine, prochlorperazine, nicotine, midazolam, acepromazine, acetaminophen, acetohexamide, acetohydroxamic acid, acetylcholine, acetylcysteine acyclovir, albendazole, alclometasone dipropionate, allopurinol, alprazolam, alprostadil, amcinoide, amantadine, amidinocillin, amikacin amiloride, aminocaproic acid, aminophylline, aminosalicylate, aminosalicylic acid, amitriptyline hydrochloride, ammonium chloride, amobarbital, amodiaquine hydrochloride, amoxapine, amoxicillin, amphetamine sulfate, amphotericin, ampicillin amprolium, acetazolamide acetyldigoxin, acetylsalicylic acid, anileridine, anthralin, antipyrine, antivenin, apomorphine, apraclonidine, ascorbic acid, aspirin, acromycin atropine, amoxycillin anipamil, azaperone azatadine maleate, azathioprine, azithromycin, aztreonam, bacampicillin, bacitracin, baclofen, barium salts, beclomethasone diproionate, belladonna extract, bendroflumethiazide, benoxinate hydrochloride, benzethonium chloride, benzocaine, benzonatate benzthiazide, benztropine mesylate, betaine, betamethasone, betaxolol, betanechol chloride, biotin, biperiden, bisacodyl, bismuth, botulism antitoxin, bromocriptine mesylate, bromodiphenhydramine hydrochloride, bumetanide, bupivacaine, busulfan butabarbital sodium, butalbital, combinations of butalbital, caffeine, beta-carotene, calcifediol, calcium carbonate, calcium citrate, calcium salts, candicidin, carbachol, carbamazepine, carbenicillin indanyl sodium, carbidopa, carbinoxamine maleate, carboprost tromethamine, carboxymethyl cellulose, carisoprodol, casanthranol, cascara, castor oil, cefaclor, cefadroxil, cefamandole nafate, cefazolin, cefixime, cefoperazone, cefotaxime, cefprozil, ceftazidime, cefuroxime axetil, cephalexin, cephradine, ceramic powder, chlorambucil, chloramphenicol, chlordiazepoxide, chloroquine phosphate, chlormadinone acetate, chlorothiazide, chlorpheniramine maleate, chloroxylenol, chlorpromazin, chlorpropamide, chlorprothixene, chlorprothixene, chlortetracycline bisulfate, chlortetracycline hydrochloride, chlorthalidone, chlorzoxazone, cholecalciferol, cholera vaccine, chromic chloride, chymotrypsin, cimetidine, cinoxazin, cinoxate, ciprofloxacin, cisplatin, clarithromycin, clavulanate potassium, clemastine fumarate, clidinium bromide, clindamycin hydrochloride, palmitate and phosphate, clioquinol, clofazimine, clofibrate, clomiphene citrate, cinnarizine, clonidine hydrochloride, clorsulon, clotrimazole, cloxacillin sodium, cyanocobalamin, cocaine, coccidioidin, cod liver oil, codeine, colchicine, colestipol, corticotropin, corisone acetate, cyclacillin, cyclizine hydrochloride, cyclobenzaprine hydrochloride, cyclophosphamide, cycloserine, cyclosporine, cyproheptadine hydrochloride, cysteine hydrochloride, danazol, dapsone, dehydrocholic acid, demeclocycline, desipramine, desoximetasone, desoxycorticosterone acetate, dexamethasone, dexchlorpheniramine maleate, dexpanthenol, dextroamphetamine, dextromethorphan, diazepam, diazoxide, dibucaine, diclofenac epolamine, dichlorphenamide, dicloxacillin sodium, dicyclomine, dienestrol, diethylpropion hydrochlorid, diethylstilbestrol, diflunisal, digitalis, dicoumarol, digitoxin, digoxin, dihydroergotamine, dihydrostreptomycin, dihydrotachysterol, dihydroxyaluminium amino acetate, dihydroxyaluminium sodium carbonate, diltiazem hydrochloride, dimenhydrinate, dimercaprol, diphenhydramine hydrochloride, diphenoxylate hydrochloride, diphteria antitoxin, dipyridamole, disopyramide phosphate, disulfuram, dobutamine hydrochloride, docusate calcium, docusate sodium, dopamine hydrochloride, doxepin hydrochloride, doxycycline, doxycycline hyclate, doxylamine cuccinate, dronabinol, droperidol, drotaverine, dydrogesterone, dyphylline, guaifenesin, enalapril maleate, analaprilat, ephedrine, equilin, ergocalciferol, ergoloid mesylates, ergonovine maleate, ergotamine tartrate, erythrityl tetranitrate, erythromycin, estradiol, estriol, estrogene, estrone, estropipate, ethcrynic acid, ethambutol hydrochloride, ethchlorvynol, ethinyl estradiol, ethionamide, ethopropazine hydrochloride, ethotoin, ethynodiol diacetate, etidronate disodium, etoposide, eugenol, famotidine, fenoprofen, ferrous fumatate, ferrous gluconate, ferrous sulfate, flucytosine, fludrocortisone acetate, flunisolide, fluocinolone acetonide, fluocinonide, fluorescein sodium, fluorometolone, fluorouracil, fluoxymesterone, fluphenazine, flurandrenolide, flurazpam, flurbiprofen, folic acid, furazolidone, flunitrazepam, furosemide, gemfibrozil, gentamicin, gentian violet, glutarate, glutethimide, glycopyrrolate, chorionic gonadotropin, gramicidin, griseofulvin, guaifenesin, guanabenz, guanadrelsulfate, halazone, haloperidol, haloprogin, halothane, heparin calcium, hepatitis virus vaccine, hetacillin potassium, hexylresorcinol, histamine phosphate, histidine, homatropine, histoplasmin, hydralazine hydrochloride, hydrochlorothiazide, hydrocodone bitartrate, hydrocortisone, hexobarbital, hydroflumethiazide, hydromorphone hydrochloride, hydroquinone, hydroxocobalamin, hydroxyamphetamine, hydroxychloroquine sulfate, hydroxyprogesterone caproate, hydroxyurea, hydroxine hydrochloride, hydroxine pamoate, hyoscyamine, hyoscyamine sulfate, ifosfamide, imipramide, imipramide hydrochloride, indapamide, indomethacin, insulin, inulin, ocetamid, iodoquinol, iohexyl, iopamidol, ipecac, ipodate calcium, ipodate sodium, isocarboxacid, isoetharine hydrochloride, isoflurane, isoniacid, isopropamide iodine, isoproterenol hydrochloride, isosorbide dinitrate, isotretenoin, isoxsuprine hydrochloride, kanamycin sulfate, ketoprofen, ketoconazole, labetalol hydrochloride, lanolin, leucine, leucovorin calcium, levamisole hydrochloride, levocamithine, levodopa, levonorgestrel, levorphanol tartrate, levothyroxine sodium, lidocaine, lincomycin hydrochloride, lindane, liothyronine sodium, liotrix, lisinopril, lithium carbonate, loperamide hydrochloride, loracarbef, lonetil, lorazepam, lovastatin, loxapine, lysine, mafenide acetate, magaldrte, magnesium carbonate, magnesiumchloride, magnesium gluconate, magnesium oxide, other magnesium salts, malathinon, manganese salts, manganese, maprotiline hydrochloride, mazindol, measle virus vaccine, mebendazole, mebrofenin, mecamylamine hydrochloride, meclizine hydrochloride, meclocycline, meclofenamate sodium, medroxyprogesterone acetate, mefenamic acid, megestrol acetate, meglumine, melphalan, menadiol sodium diphosphate, menadione, menotropine, meperidine, mephenyloin, mephobarbital, meprednisone, meprobaamate, mercaptopurine, mesoridazine besylate, mestranol, metaproterenol sulfate, metaraminol bitartrate, methacycline hydrochloride, methadone hydrochloride, methamphetamine hydrochloride, methazolamide, methdilazine, methenamine, methicillin sodium, methimazole, methionine, methocarbamol, methotrexate, methoxsalen, methoxyflurane, methsuximide, methyclothiazide, methylbenzethonium chloride, methyldopa, methylergonovine maleate, methylphenidate hydrochloride, methylprednisolone, methyltestosterone, methysergide maleate, metoclopramide, metolazone, meoprolol tartrate, metronidazole, metyrapone, metyrosine, mexiletine hydrochloride, mexiletine hydrochloride, miconazole, minocycline hydrochloride, minoxidil, mitomycin, mitotane, molindone hydrochloride, monobenzone, morphine sulfate, mupirocin, medazepam, mefruside, methandrostenolone, methylsulfadiazine, nadolol, nafcillin, nafcillin sodium, nalidixic acid, nalorphine, nandrolone decanoate, nandrolone phenpropionate, naproxen, natamycin, neomycin, neomycin sulfate, neostimine bromide, niacin, nitrofurantoin, nalidixic acid, nitrazepam, nitrofurantoin, nitromerson, nizatidine, nonoxynol-9, norethindrone, norethindrone acetate, norfloxacin, norgestrel, nortriptyline hydrochloride, noscapine, novobiocin sodium, nystatin, opium, oxacillin sodium, oxamniquine, oxandrolone, oxazepam, oxprenolol hydrochloride, oxtriphylline, oxybenzone, oxybutynin chloride, oxycodone hydrochloride, oxycodone, oxymetazoline hydrochloride, oxymetholone, oxymorphone hydrochloride, oxyphenbutazone, oxytetracycline, padimate, panreatin, pancrelipase, papain, panthenol, papaverin hydrochloride, parachlorophenol, paramethasone acetate, paregoric, paromomycin sulfate, penicillamine, penicillin, penicillin derivatives, pentaerythritol tetranitrate, pentazocine, pentazocine hydrochloride, pentazocine salts, pentobarbital sodium, perphenazine, pertussis, phenacemide, phenazopyridine hydrochloride, phendimetrazine tartrate, phenelzine sulfate, phenmetrazine hydrochloride, phenobarbital, phenophtalein, phenoxybenzamine hydrochloride, phentermine hydrochloride, phenylalanine, phenylbutazone, phenylephrine hydrochloride, phenylpropanolamine hydrochloride, physostigmine, phytonadione, pilocarpine, pimozide, pindolol, piperazine, piroxicam plicamycin, poliovirus vaccine inactivated, polycarbophil, polymycin b sulfate, polythiazide, potassium chloride, potassium citrate, potassium cluconate, potassium iodine, potassium sodium tartrate, povidone iodine, pralidoxime chloride, pramoxine hydrochloride, pramezam, prazepam, praziquantel, prazosin hydrochloride, prazosin hydrochloride, prednisolone, prilocaine, primaquine, primidone, probenecid, probucol, procainamide hydrochlorid, procaine hydrochloride, procarbacine hydrochloride, prochlorperazine maleate, procyclidine hydrochloride, progesterone, proline, promazine, promazine hydrochloride, promazine, promethazine, promethazine hydrochloride, propafenone hydrochloride, propantheline, proparacaine hydrochloride, propoxycaine hydrochloride, propoxyphene hydrochloride, propoxyphene napsylate, propanolol hydrochloride, propyliodone, propylthiouracil, propylthiouracil, protriptyline hydrochloride, pseudoephedrine hydrochloride, pumice, pyrantel pamoate, pyrazinamide, pyrethrum extract, pyridostigmine bromide, pyridoxine hydrochloride, pyrilamine maleate, pyrimethamine, pyroxylin, pyrvinium pamoate, phenacetin, phenyloin, prednisone, uinidine gluconate, quinidine sulfate, rabies vaccine, racepinephrine ranitidine, rauwolfia serpentina, resorcinol, ribavirin, riboflavin, rifampin, ritodrine, rubella virus vaccine, saccharin, saccharin sodium, salicylamide, salsalata, secobarbital sodium, selenius acid, selenium sulfate, sennaserine, simethicone, sodium ascorbate, sodium bicarbonate, sodium fluoride, sodium gluconate, sodium iodide, sodium lactate, sodium nitrite, sodium ditroprusside, sodium salicylate, spironolactone, stannozolol, streptomycin, sucralfate, sulfacetamide, sulfadiazine, reserpine, sulfadioxine, sulfamerazine, sulfamethazine, sulfamethizole, sulfamethoxazole, sulfamethoxydiazine, sulfapyridin, sulfasalazine, sulfaperin, sulfathiazole, sulfisoxazole, sulfinpyrazone, sulindac, suprofen, stilains, tamoxifen citrate, taurine, temacepam, terbutaline sulfate, terfenadine, terpin, testolacton, testosterone, tolazamide, tolbutamide, tetracaine, tetracycline, tetrahydrocycline, theophylline, thiabendazole, thiamine hydrochloride, thiamin, thiamylal, thiethylperazine thimerosal, thioguanine, thioridazine hydrochloride, thistrepton, thiotepa, thiothixene, threonine, thyroid, ticarcillin, timolol, tioconazole, titaniumdioxide, tutanium powder, tolazamide, tolbutamide, tolmetin, tolnaftate, trazodone hydrochloride, tretinoin, triacetin, triamcinolone, triamterene, triazolam, trichorfon, trichlonnethiazide, trientine hydrochloride, trifluoperazine hydrochloride, triflupromazine, trihexyphenidyl hydrochloride, trimeprazine tartrate, trimethadione, trimethobenzamide hydrochloride, trimethoprim, trioxsalen, tripelennamine, triprolidine, trisulfapyrimidine, tropicamide, trypsin, tryptohan, tuberculin, tyloxapol, tyropanoate sodium, tyrosine, tyrothricin, thyrothricin bethamethasone, thiotic acid, sotalol, salbutamol, norfenefrine, silymarin, dihydroergotamine, buflomedil, etofibrate, indometacin, urea, valine, valproic acid, vancomycin hydrochloride, vasopressin, verapramil, vidarabine, vinblastine, vincristine, vitamins, warfarin, yellow fever vaccine, zinc acetate, zinc carbonate, zinc chloride, zinc gluconate, beta acetyl digoxin, piroxicam, haloperidol, ISMN, amitriptylin, diclofenac, nifedipine, verapamil, pyritinol, nitrendipin, doxycycline, bromhexine, methylprdnisolone, clonidine, fenofibrate, allopurinol, pirenyepine, levothyroxin, tamoxifen, metildigoxin, o-(beta-hydroxyethyl)-rutoside, propicillin, aciclovir mononitrate, paracetamol, naftidrofuryl, pentoxifylline, propafenone, acebutolol, L-thyroxin, tramadol, bromocriptine, loperamide, ketotifen, fenoterol, cadobelisate, propanolol, enalaprilhydrogen maleate, bezafebrate, ISDN, gallopamil, xantinol nicotinate, digitoxin, flunitrazepam, bencyclane, dexapanthenol, pindolol, lorazepam, diltiazem, piracetam, phenoxymethylpenicillin, furosemide, bromazepam, flunarizin, erythromycin, metoclopramide, acemetacin, ranitidin, biperiden, metamizole, doxepin, dipotassium chloroazepate, tetrazepam, estramustine phosphate, terbutaline, captopril, maprotiline, prazosin, atenolol, glibenclamide, cefaclor, etilfrine, cimetidine, theophylline, hydromorphone, ibuprofen, primidone, clobazam, oxaceprol, medroxyprogesterone, flecainid, pyridoxal-5-phosphate glutaminate, hymechromone, etofylline clofibrate, vincamine, cinnarizine, diazepam, ketoprofen, flupentixol, molsimine, glibornuride, dimetinden, melperone, soquinolol, dihydrocodeine, clomethiazole, clemastine, glisoxepide, kallidinogenase, oxyfedrine, baclofen, carboxymethylcysteine, thioridazine, betahistine, L-tryptophan, murtol, bromelaine, prenylamine, salazosulfapyridine, astemizol, sulpiride, benzerazide, dibenzepine, acetylsalicylic acid, miconazol, nystatin, ketoconazole, sodium picosulfate, coltyramine, gemfibrocil, rifampicin, fluocortolone, mexiletin, amoxicillin, terfenadrin, mucopolysaccharide polysulfade, triazolam, mianserin, tiaprofenic acid, amezinium metilsulfate, mefloquine, probucol, quinidine, carbamazepine, L-aspartate, penbutolol, piretanide, aescin amitriptyline, cyproterone, sodium valproinate, mebeverine, bisacodyl, 5-aminosalicylic acid, dihydralazine, magaldrate, phenprocoumon, amantadine, naproxen, carteolol, famotidine, methyldopa, auranofine, estriol, nadolol, levomepromazine, doxorubicin, medofenoxate, azathioprine, flutamide, norfloxacin, fendiline, prajmalium bitartrate, lipid derivatives of phosphonatides, amphiphilic polymers, adenosine derivatives, sulfated tannins, monoclonal antibodies, and metal complexes of water soluble texathyrin. Additional drugs are contemplated such that this list is not intended to be exhaustive or comprehensive.

Hormones suitable for buccal absorption are the insulins (e.g., human insulin, bovine insulin, porcine insulin, and biosynthetic human insulin including Humulin®), somatostatin, vasopressin, calcitonin, estrogen, progestin, testosterone, glucagon, glucagon-like peptide (GLP-1) and its analogs, for example. The active ingredient may also be a protein, enzyme, a peptide, a polysaccharide, a nucleic acid, a cell fragment, a biologically active substance, a salt, or the like. The active agent may also be a lipid such as, but not limited to, fat-soluble vitamins (e.g., vitamins A, D, E and K), ceramides in which the fatty acid components may be one or more of the following: alpha-hydroxy 6-hydroxy-4-sphingenine, alpha-hydroxy phytosphingosine, alpha-hydroxy sphingosine, ester linked omega-hydroxy 6-hydroxy-4-sphingenine, non-hydroxy phytosphingosine, non-hydroxy sphingosine, and/or ester linked omega-hydroxysphingosine and free sterols.

One or more cannabinoids may also be delivered. The term “cannabinoid” as used herein, refers to a class of diverse chemical compounds that acts on cannabinoid receptors in cells that alter the neurotransmitter response in the brain. Ligands for these receptor proteins include the endocannabinoids (produced within the body by animals), the phytocannabinoids (found in cannabis and some other plants), and synthetic cannabinoids (not present in nature). The most notable cannabinoids are tetrahydrocannabinol (THC, the primary psychoactive compound in cannabis), cannabidiol (CBD, the non-psychoactive compound in cannabis), and their acidic forms of tetrahydrocannabinolic acid (THCA) and cannabidiolic acid (CBDA). Other notable cannabinoids are again either found in their decarboxylated forms and acidic forms, such as cannabigerol (CBG) and cannabigerolic acid (CBGA), cannabichromene (CBC) and cannabichromenic acid (CBCA). Each of these decarboxylated and acidic forms have corresponding homologs in which the propyl (3-carbon) side chain is present in place of a pentyl (5-carbon) side chain on the compound. For the four most notable cannabinoids, the corresponding decarboxylated and acidic forms are as follows: tetrahydrocannabivarin (THCV), tetrahydrodrocannabivarin carboxylic acid (THCVA), cannabidivarin (CBDV), cannabidivarin carboxylic acid (CBDVA), cannabigerovarin (CBGV), cannabigerovarin carboxylic acid (CBGVA), cannabichromevarin (CBCV), cannabichromevarin carboxylic acid (CBCVA). Other notable cannabinoids which are chemically derived products of the notable cannabinoids include cannabinol (CBN), cannabicyclol (CBL), cannabivarin (CBV). Other minor cannabinoids include cannabigerol monomethyl ether (CBGM), cannabielsoin (CBE), and cannabicitran (CBT), cannabicylolic acid (CBLA), cannabicyclovarin (CBLV), cannabidiorcol (CBD-C1), cannabigerolic acid monomethyl ester (CBGAM), cannabinodiol (CBND), cannabinol methylether (CBNM), cannabinol-C2 (CBN-C2), cannabinol-C4 (CBN-C4), cannabinolic acid (CBNA), cannabiorcol (CBN-C1), 10-ethoxy-9-hydroxy-delta-6a-tetrahydrocannabinol, 8,9-dihydroxy-delta-6a-tetrahydrocannabinol, cannabitriol, cannabitriolvarin (CBTV), delta-8-tetrahydrocannabinol (Δ8-THC), delta-8-tetrahydrocannabinol (Δ8-THCA), tetrahydrocannabinol-C4 (THC-C4), tetrahydrocannabinolic acid B (THCA-B), tetrahydrocannabinolic acid-C4 (THCA-C4), tetrahydrocannabiorcol (THC-C1), tetrahydrocannabiorcolic acid (THCA-C1), 10-oxo-delta-6a-tetrahydrocannabinol (OTHC), cannabichromanone (CBCF), cannabifuran (CBF), cannabiglendol, cannabiripsol (CBR), dehydrocannabifuran (DCBF), cis-tetrahydrocannabinol (cis-THC), tryhydroxy-teterahydrocannabinol (triOH-THC), cannabinerolic acid, cannabidiol monomethyl ester (CBDM), cannabidiol-C4 (CBD-C4), cannabinovarin (CBNV), (−)-(9 R, 10 R)-trans-cannabitriol, (+)-(9S,10S)-cannabitriol, (±)-(9R,10S/9S,10R)-cannabitriol, (−)-(9R,10R)-trans-10-O-ethyl-cannabitriol, (±)-(9R,10R/9S,10S)-cannabitriol-C3, cannabiolic acid cannabitriol, (−)-6a,7,10a-trihydroxy-tetrahydrocannabinol, CBDA-9-OH-CBT-C5 ester, (−)-cannabitetrol, 5aS,6S,9R,9aR)-cannabielsoin, (5aS,6S,9R,9aR)-C3-cannabielsoin, (5aS,6S,9R,9aR)-cannabielsoic acid A, (5aS,6S,9R,9aR)-cannabielsoic acid B, (5aS,6S,9R,9aR)-C3-cannabielsoic acid B, cannabiglendol-C3, (−)-Δ7-trans-(1R,3R,6R)-isotetrahydrocannabinol, (±)-Δ7-1,2-cis-(1R,3R,6S/1S,3S,6R)-isotetrahydrocannabivarin, (−)-Δ7-trans-(1R,3R,6R)-isotetrahydrocannabivarin, cannabichromanone-C3, cannabicoumaronone, and 3,4,5,6-tetrahydroxy-alpha-2-trimethyl-9-n-propyl-2,6-methano-2H-1-benzoxocin-5-methanol. Other cannabinoids from nature not derived from cannabis plants include alykylamides, catechins, beta-caryophyllene, anandamide, 7,10,13,16-docosatetraenolyethanolamide, homo-γ-linolenoylethanolamine, N-acylethanolamines, 2-arachidonoylglycerol, 2-arachidonyl glyceryl ether, N-arachidonoyl dopamine (NADA), virodhamine (OAE), and lysophosphatidylinositol (LPI). Notable cannabinoids which are synthetic and thus not derived from nature include marinol, cesamet, SR141716, JWH-018, JWH-073, CP-55940, HU-210, HE-331, SR144528, WIN 55,212-2, JWH-133, nantrodolum, and AM-2201.

One or more than one of the foregoing active ingredients, may be combined together in a single pharmaceutical formulation or administered serially.

Buccal administration (i.e., via the pouch of the cheek of the patient) is particularly useful for active therapeutic agents which show poor bioavailability upon administration through other non-parenteral modes. It is necessary for a buccal composition to remain in contact with the oral mucosa 14 for a time sufficient for absorption of the medicament to be administered. If the formulation falls apart too quickly, the active ingredient may be swallowed, and an insufficient amount of medicament is delivered transbuccally. The composition should be of a small size and it is desirable that as much of the composition as possible not be diluted by or soluble in saliva.

The oral mucosa 14 has a rich vasculature 18 that makes it suitable route of administration for delivery of life-saving emergency medicines into the bloodstream of a subject in need of treatment. The present invention provides devices and methods for rapid transmucosal or transbuccal delivery of a pharmaceutical formulation that adheres to mucous membranes and rapidly delivers a pharmaceutically active ingredient such as epinephrine or naloxone. Such pharmaceutical formulations can include an effective amount (e.g., a unit dose) of the active ingredient, a resin or other film-forming ingredient, a volatile solvent, and optionally water. When applied to the buccal membrane 13, for example, these pharmaceutical compositions can achieve a rapid and early onset of a therapeutic effect necessary to save a patient's life. The applicator is intended for a single use, then properly discarded. It is also designed for use by persons who lack medical training (i.e., a layperson).

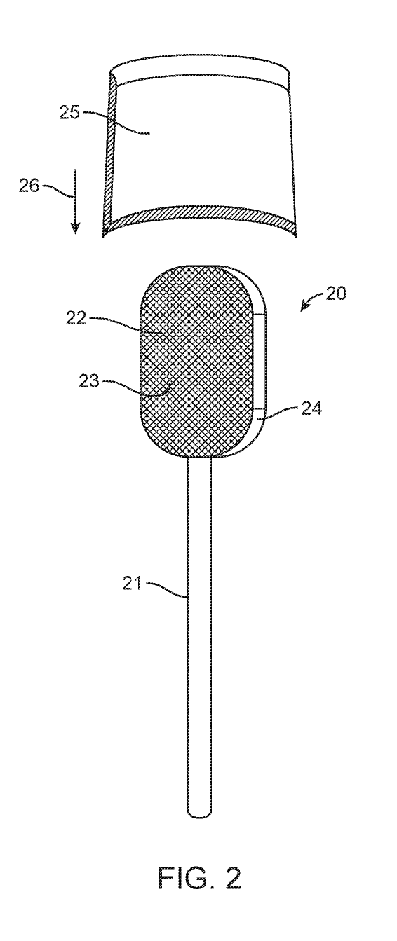

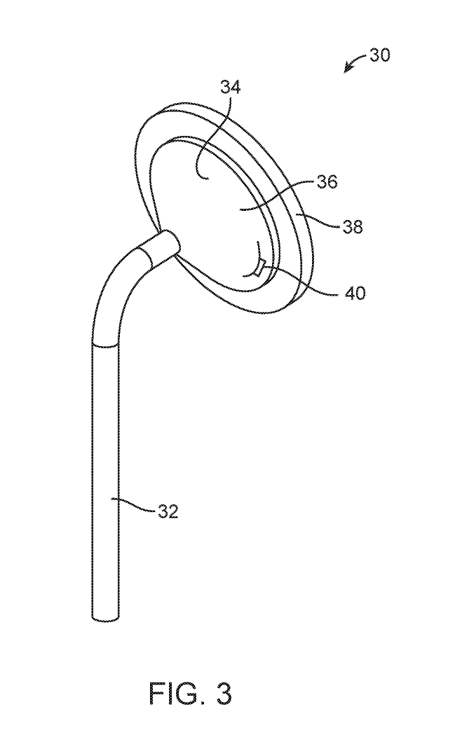

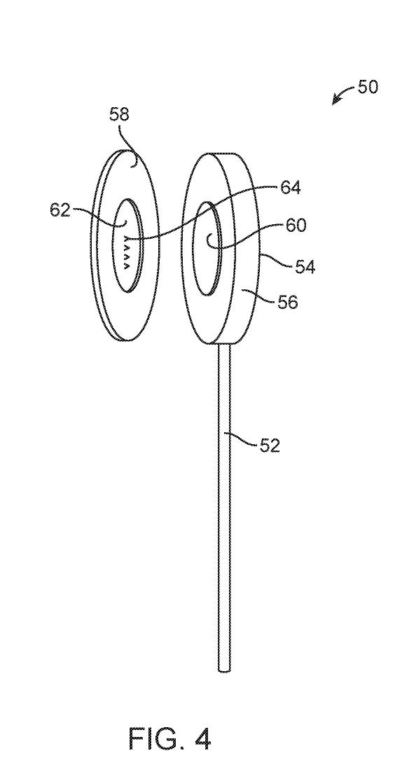

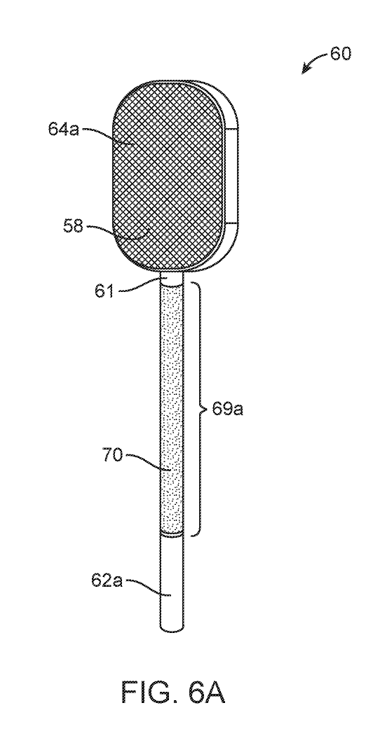

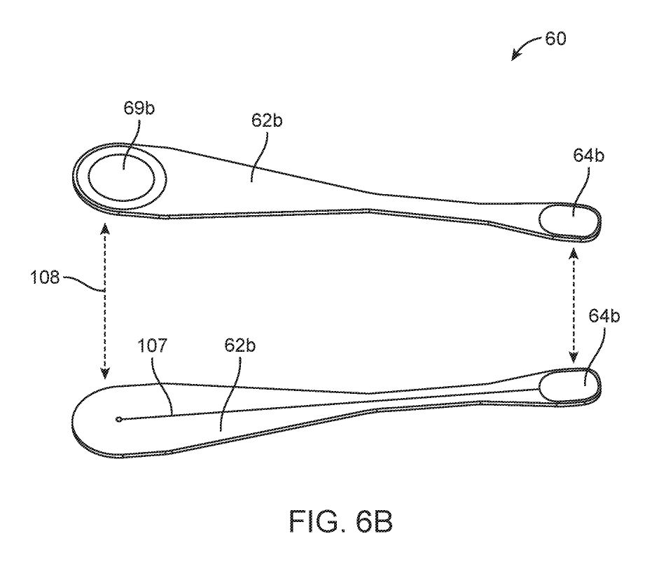

Turning now to FIG. 2, the device 20 includes a handle 21 and an applicator tip 22 that is inserted into the mouth of a patient. The handle 21 may be any length, diameter, or configuration sufficient to allow a caregiver (i.e., user) to grasp the handle 21 of the device 20 with their fingers and position the applicator tip 22 in the oral cavity 1 of the person requiring medical attention without the fingers of the caregiver contacting the teeth or oral tissues 4, 5, 6, 7 of the patient. Advantageously, this provides an easy-to-use device that prevents the patient from biting the caregiver's fingers and eliminates the transmission of saliva, mucus, blood, vomitus, or other bodily fluids between the caregiver and the person requiring medical assistance. The handle 21 also allows easy removal of the applicator tip 22 from the patient's mouth in the event of choking, vomiting, or concluding successful treatment (e.g., resuscitation), for example. The likelihood that the caregiver will not hesitate to provide (often urgent) care is increased for at least these reasons. The applicator tip 22 and application layer 23 may be any shape, size or configuration that allows easy placement in the oral cavity with sufficient surface area contacting the oral tissue to allow efficient drug delivery. The shape of the applicator tip 22 may be rectangular with rounded corners (FIG. 2), circular (FIG. 3), or oval (FIG. 6B), for example. Many other shapes are contemplated such that this disclosure is not limited to these exemplary shapes.

According to one embodiment, the surface of the applicator tip 22 has abrasive properties. When gently rubbed on the oral mucosal surface, the applicator tip 22 increases the permeability of an area of the mucosal epithelial cell layer 15 physically (e.g., by rubbing or abrading) or by application of a chemical permeation enhancer, which can accelerate systemic absorption of an active ingredient and significantly improve the rapidity of drug delivery in emergency situations.

In one version, the applicator contains epinephrine for the emergency treatment of anaphylaxis. In another version, the applicator contains naloxone for the emergency treatment of an opioid overdose.

In each case, the applicator is placed in the mouth of the patient with the drug-containing side of the applicator tip facing the buccal area 7 of the oral cavity 1 or the upper or lower lips 2a, 2b to apply the drug.

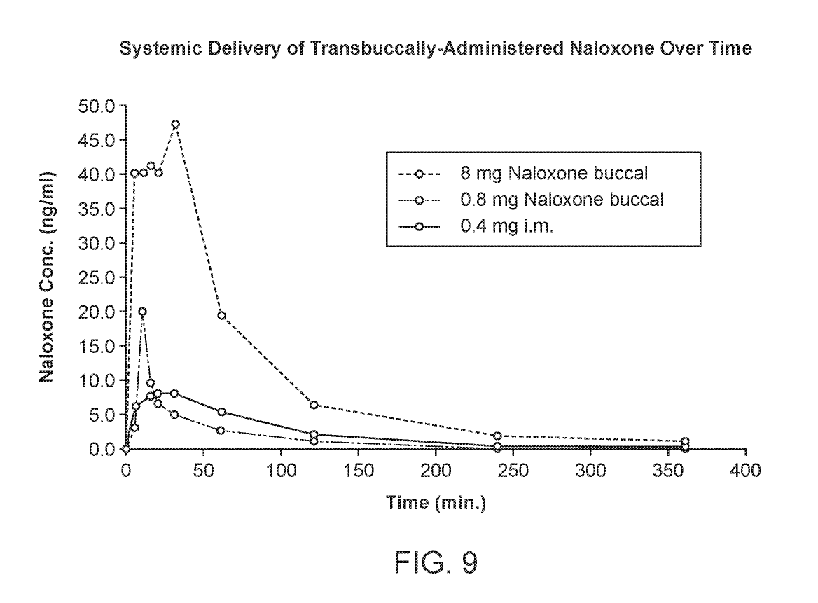

The time for the active ingredient to reach its maximum plasma concentration in the bloodstream is referred to as the Tₘₐₓ. The method results in a Tₘₐₓ of the active ingredient of less than 30 minutes. (FIG. 9). For epinephrine, delivery of 3.0 or 30.0 mg when applied via a film-forming resin results in Cₘₐₓ plasma levels between 0.1-5.0 ng/ml or 1.0-50 ng/ml respectively. For naloxone, delivery of 0.4 or 4.0 mg results in Cₘₐₓ plasma levels between 1-10 ng/ml or 10-1000 ng/ml, respectively.

Delivery Device

The delivery device 20 includes a handle 21 and an applicator tip 22. Two embodiments are depicted in FIGS. 2, 3 and 4 as described in related U.S. Provisional Patent Application Ser. No. 62/526,251, filed Jun. 28, 2017, entitled “Apparatus and Methods for Rapid Transbuccal Drug Delivery” the disclosures of which are hereby incorporated by reference in their entirety for all purposes:

Single Compartment Configuration: Drug Contained within the Applicator Tip

This configuration depicts a generally oval-shaped applicator tip 22 and a plastic handle 21. The applicator tip 22 has a single-compartment design and includes a rigid or semi-rigid backing 24 and an application layer 23 that is attached to the backing 24. The application layer 23 is pre-loaded with a suitable amount of a pharmaceutical formulation that includes an effective amount of the active ingredient.

The application layer 23 is made of the following: 1) A hook-and-eye fastener material (e.g., Velcro®) with a rigid or semi-rigid solid, non-porous plastic backing. This module is covered with a protective colored plastic shield to keep the drug protected from air and light. 2) Plastic bristles (Guangzhou City Nansha Ming Wang Synthetic Fiber Factory), polyester filament (Shanghai Xiaobang Household Products Co., Ltd.), or any other non-reactive, shelf-stable, substance known by those of skill in the art to be both highly absorbent and also able to release absorbed material onto a substrate such as the oral mucosa 14. Of course, many other substances known by those of skill in the art are contemplated including, but no limited to, materials generally recognized as safe (GRAS) as designated by the American Food and Drug Administration (FDA). This module is covered with a protective shield 25 to keep drug protected from air and light. The shield 25 may be ridged or flexible and may be colored, transparent or opaque. In one embodiment, the protective shield 25 is an amber-colored plastic.

Per FIG. 2, the applicator tip 22 (or the entire device 20) is covered with a protective plastic shield 25. The protective shield 25 may be slid over the application tip 22 or over the entire device 20 in direction 26 or any other direction. The shield 25 may form a hermetic seal around the application tip 22 or around the entire device 20, for example. The device 20 may be sterilized before being sealed with the protective shield 25.

In order to use the device 20, the user removes the protective plastic shield 25 surrounding the applicator tip 22 or the entire device 20 to expose the application layer 23 that contains the pharmaceutical formulation. Then, the user grasps the handle 21 and inserts the applicator tip 22 into the mouth of the patient with one hand while holding back upper lip 2a, lower lip 2b, cheek 3, or the corner of the mouth with the other hand. With the application layer 23 facing the inner tissue of upper or lower lips 2a, 2b or inside the cheeks 3, the user may rub the application layer 23 gently back and forth a few times along oral mucosa (i.e., inside cheek area, or under upper and lower lips) to ensure drug is spread over an area of at least 1-2 cm². Optionally rubbing or abrading the mucosal tissue causes more rapid absorption of the active ingredient. Then, the user removes the applicator tip 22 from patient's mouth and discards the device. If the desired effect is not observed after 2-10 minutes, the user repeats the procedure using a second unused (i.e., new) device 20. In the case of opioid overdose, the desired effect may include the patient returning to a normal unaided breathing pattern and/or a regular heart rhythm. Naloxone is active in the body for about 30-90 minutes, and its therapeutic effects could wear off before those of the opioids, causing the return of serious symptoms indicative of drug overdose. In the case of anaphylactic shock, the desired effect may include increased blood pressure and/or more efficient breathing. Epinephrine metabolizes in the liver and kidneys relatively quickly. Therefore, the sequence of steps described above can be repeated multiple times as treatment requires. After the pharmaceutical formulation (e.g., naloxone, epinephrine, etc.) is administered, the patient should be carefully monitored while emergency personnel are summoned.

Dual Compartment Configuration with Drug Release Tab

As shown in FIG. 3, the device 30 includes a rigid or semi-rigid plastic handle 32 and an applicator tip 34 that is formed in or attached to the handle 32 according to another embodiment of the invention. The handle 32 may be substantially straight (FIGS. 2, 4, 5A-5B), bent (FIG. 3), or curved to facilitate placement against the mucosa.

Applicator tip 34 includes two separate, isolated compartments, a drug reservoir 36 that holds a pharmaceutical formulation, which includes an effective amount of an active ingredient, and a nonreactive, porous, application layer 38, such as a foam, flocked polyester, or cotton, that optionally has an abrasive surface. Drug reservoir 36 is isolated from application layer 38 by a nonporous barrier (not shown), such as a pierceable membrane, that prevents fluid flow from drug reservoir 36 to application layer 38, unless the barrier is displaced or disrupted. According to one embodiment, a drug release tab 40 is attached to the nonporous barrier. When pulled, the release tab 40 displaces or disrupts (e.g., tears or pierces) the membrane, permitting the pharmaceutical formulation to enter and gorge the application layer 38 with the pharmaceutical formulation. The drug reservoir 36, which is attached to handle 32, may be cone-shaped and contains the pharmaceutical formulation. Drug reservoir 36 is optionally compressible such that, using finger pressure, the user can force the pharmaceutical formulation from the drug reservoir 36 into the application layer 38.

In order to use the device, the user removes the protective plastic shield surrounding the applicator tip 34. Then, the drug-release tab 40 is pulled, piercing the membrane that separates the drug reservoir 36 and application layer 38. The drug product held in drug reservoir 36 is thus permitted to gorge the application layer 38. The user grasps the handle 32 and inserts applicator tip 34 into the mouth of the patient with one hand while holding back the upper lip, lower lip, cheek, or corner of the mouth of the patient with the other hand. With the application layer 38 facing the inner tissue of upper or lower lips, inside the cheeks, or the buccal region, the user rubs the surface of the application layer 38 gently back and forth a few times along the oral mucosa to ensure that the pharmaceutical composition is spread over an area of at least 1-2 cm² and to rub or abrade the mucosal tissue to cause more rapid absorption of the active ingredient. Then, the user removes the device from the patient's mouth and discards. If the patient is not revived after 2-10 minutes, the user needs to repeat the procedure while using a second unused device 30. This sequence of steps can be repeated multiple times as treatment requires.

Dual Compartment Configuration with Spiked Button

As shown in FIG. 4, the device 50 includes a rigid or semi-rigid plastic handle 52 and an applicator tip 54 that is formed in or attached to the handle 52 according to yet another embodiment of the invention.

Applicator tip 54 includes two separate, isolated compartments, a drug reservoir 56 that holds a pharmaceutical formulation, which includes an effective amount of an active ingredient, and a nonreactive, porous, application layer 58, such as a foam that optionally has an abrasive outer surface. A cellophane cover (not shown) covers the outer surface of the application layer 58 (or the entire applicator tip 54). A nonporous pierceable barrier or membrane 60, is positioned between the drug reservoir 56 and the application layer 58, and, when intact, prevents fluid flow from drug reservoir 56 to application layer 58. A porous button 62, which is attached to the inner surface of application layer 58, is configured to include spiky projections 64 that extend toward the nonporous barrier 60.

In order to use the device, the user presses on the middle of the cellophane cover (which optionally can be provided with a painted red circle as a visual guide). Doing so causes the spiky projections 64 of button 62 to pierce the membrane 60 and allow the pharmaceutical formulation to enter the application layer 58. Holding the device 50 with the application layer facing down for a few seconds ensures that the pharmaceutical formulation fills the application layer 58. Alternatively, if the drug reservoir 56 is compressible, finger pressure on the exterior of the drug reservoir 56 can push the pharmaceutical formulation into the application layer 58. The user then removes the cellophane cover and applies the pharmaceutical formulation to the oral mucosa. If the patient is not revived after about 2-10 minutes, the user needs to repeat the procedure while using another unused device 50. Again, this sequence of steps can be repeated multiple times as treatment requires.

Dual Compartment Configuration in Handle Design

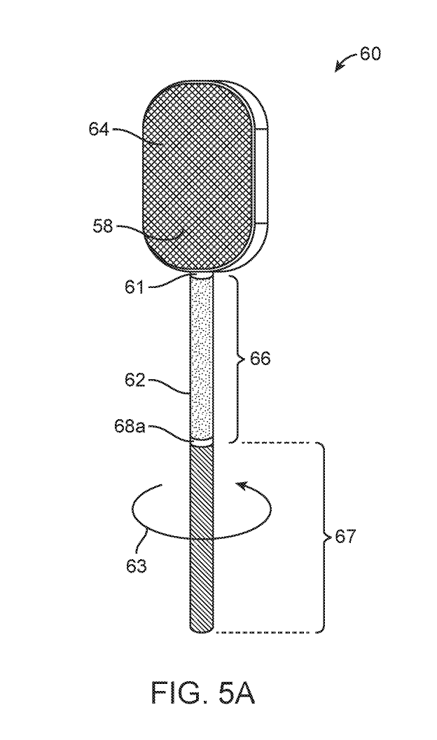

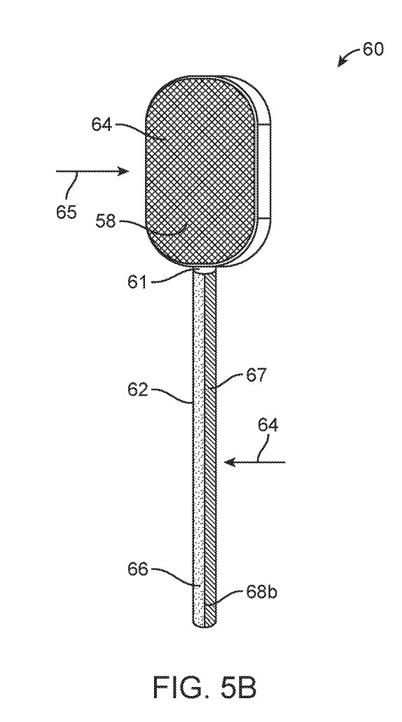

As shown in FIGS. 5A-5B, the device 60 includes a rigid or semi-rigid plastic handle 62 and an applicator tip 64 that is formed in or attached to the handle 62 according to still another embodiment of the invention.

The handle 62 includes two separate, isolated compartments. Each compartment includes: 1) a drug reservoir 66 that holds a pharmaceutical formulation, which includes an effective amount of an active ingredient; and 2) a diluent reservoir 67 containing a diluent. These reservoirs, contained inside the handle, may occupy most, all, or a fraction of the handle. The handle 62 may be of any length, shape or other configuration so as to allow access to the oral mucosa without the caregiver contacting the inside of the patient's mouth. The reservoirs are separated by a partition. The partition 68a may be configured in a perpendicular orientation relative to the handle length as shown in FIG. 5A. Alternatively, the partition 68b may be configured in a vertical orientation along a length of the handle as shown in FIG. 5B, for example. Other configurations (e.g., diagonal) are, of course, possible. The partition may be constructed of any breakable non-reactive substance including glass or plastic, for example.

Applicator tip 64 includes a non-reactive, porous, application layer 58, such as a foam that optionally has an abrasive outer surface. A plastic wrap (e.g., Cellophane® or polyvinyl chloride) cover 25 (FIG. 2) surrounds the outer surface of the application layer 58 (or the entire applicator tip 64 or the entire device 60). In addition to the nonporous partition or barrier 68a or 68b positioned between the drug reservoir 66 and the diluent 67, a partition 61 separates the drug reservoir 66 (and diluent reservoir 67) from the applicator tip 64 and, when intact, prevents fluid flow from drug reservoir 66 (and diluent reservoir 67) to the application layer 58. The partition can be positioned at any location along the length of the handle provided the partition separates the drug reservoir 66 and diluent reservoir 67 from the application tip 64 before use.

The partitions 61 and 68a or 68b can be broken by twisting the handle 62 of the device 60 in direction 63, for example. Alternatively or additionally, bending the handle in directions 64 and 65 per FIG. 5B may also be employed to break barriers 61 and 68a or 68b. Doing so causes the active ingredient and the diluent to mix and flow into the applicator tip 64. Shaking or gently agitating the device allows the active ingredient to thoroughly mix with the diluent. Holding the device 60 with the application layer 58 facing downward for a few seconds uses gravity to help ensure that the pharmaceutical formulation containing the active ingredient gorges the application layer 58.

Alternatively, if the drug reservoir 66 is compressible, finger pressure on the exterior of the drug reservoir 66 can push the pharmaceutical formulation into the application layer 58. A similar method can be used to push the diluent in contact with the active ingredient. Then the user removes the cellophane cover and applies the pharmaceutical formulation to the oral mucosa (upper and lower lips and/or buccal region). If the patient is not revived after 2-10 minutes, the user needs to repeat the procedure while using a second unused device 60. This sequence of steps can be repeated multiple times as treatment requires.

Single Compartment Configuration in Handle

As shown in FIG. 6A, the device 60 includes a rigid or semi-rigid plastic handle 62a and an applicator tip 64a that is formed in or attached to the handle 62a according to another embodiment of the invention.

The handle 62a includes one compartment containing a reservoir 69a holding a pharmaceutical formulation. In contrast to the embodiment depicted in FIGS. 5A, and 5B, the pharmaceutical formulation described in this embodiment includes an effective amount of an active ingredient (i.e., drug) and diluent premixed in solution. This reservoir, contained inside the handle, may occupy most, all, or a fraction of the handle. The handle 62a may be of any length, shape or other configuration so as to allow access to the oral mucosa without the caregiver contacting the inside of the patient's mouth.

The reservoir 69a and applicator tip 64a are separated by a partition 61 and, when intact, prevents fluid flow from reservoir 69a to the application layer 58. The partition 61 may be configured in a perpendicular orientation relative to the handle length as shown in FIG. 6A. Of course, other configurations, including diagonal, convex, concave, etc., are possible. The partition may be constructed of any breakable non-reactive substance including glass or plastic, for example.

Applicator tip 64a includes a non-reactive, porous, application layer 58, such as a foam that optionally has an abrasive outer surface. A cellophane cover 25 (FIG. 2) covers the outer surface of the application layer 58 (or the entire applicator tip 64a) to form a hermetic seal.

The partition 61 may be broken by twisting or bending the handle 62a of the device 60 as previously described with respect to FIGS. 5A and 5B. Breaking the partition causes the pharmaceutical formulation to flow from the reservoir 69a into the applicator tip 64a. Holding the device 60 with the application layer 58 facing downward for a few seconds uses gravitational force to help fill the application layer 58 with the pharmaceutical formulation 70. Alternatively, if the drug reservoir 69a is compressible, finger pressure on the exterior of the drug reservoir 69a can push the pharmaceutical formulation 70 into the application layer 58. Then the user removes the cellophane cover and applies the pharmaceutical formulation 70 to the oral mucosa (upper and lower lips and/or buccal region). If the patient is not revived after 2-10 minutes, the user needs to repeat the procedure while using a second new device 60. This sequence of steps can be repeated multiple times as treatment requires.

Maintaining the pharmaceutical formulation 70 separate from the applicator tip 64a until administration to the patient may also be accomplished using existing, modified or customized versions of Color Ring™ Swab Applicator or XPRESS™ Applicators from Swabplus, Inc. (www.swabplus.com/technology), for example.

FIG. 6B depicts an alternative embodiment of the invention wherein the pharmaceutical formulation 70 is contained in a reservoir 69b located in the handle and enclosed by a breakable blister, bleb, or other compartment-based compliant packaging. The blister may be made of thermal bonded foil, adhesive, or similar material that prevents degradation of the pharmaceutical by ultraviolet light, prohibits evaporation (particularly with regard to ethanol-based formulations), increases shelf life and/or sustains drug stability. Examples of blister packaging technology include ThinXXS Microtechnology, J-Pac Medical, McKesson Corporation, and others known to those of ordinary skill in the art. The applicator tip 64b may be made partially or completely from a flocked polyester pad material, cotton, a polytetrafluoroethylene (PTFE) material, a polyethersulfone (PES) material, or other similar material. The material is capable of absorbing the formulation when it is released from the reservoir and even more capable of allowing the formulation to release from the applicator tip onto the surface of the mucosa during use.

The formulation is release from the blister when the blister is broken via applied pressure such as squeezing the blister between fingers, for example. Disrupting the blister breaks the barrier and permits flow of the pharmaceutical agent from the internal reservoir 69b in the handle to the applicator tip 64b by gravity or capillary action along a channel 107 that connects the drug reservoir 69b to the pad of the applicator tip 64b.

A two-part joined configuration is envisioned for ease of manufacture. As shown in exploded view in FIG. 6B, the first part of the device 60 includes a channel 107 in a first half of the handle 62b, and an applicator tip 64b. The second part includes the second half of the a rigid or semi-rigid plastic handle 62b and an applicator tip 64b that is formed in or attached to the handle 62b. The handle 62 includes one compartment containing a reservoir 69b holding a pharmaceutical formulation. Similar to FIG. 6A, the pharmaceutical formulation described in this embodiment includes an effective amount of an active ingredient (i.e., drug) and diluent premixed in solution. This reservoir 69b, contained inside a blister or bleb in the handle, may occupy a fraction of the handle. The handle 62 may be of any length, shape or other configuration so as to allow access to the oral mucosa without the caregiver contacting the inside of the patient's mouth. The first and second molded parts of the device shown in FIG. 6B may be joined by hinge, clip, or other mechanism to fasten the first and second parts together during manufacturing as shown along direction 108, for example. Fastening the parts together encloses the canal within the middle of the device to effectively connect the reservoir 69b and applicator tip 64b.

Diluent in Handle Configuration

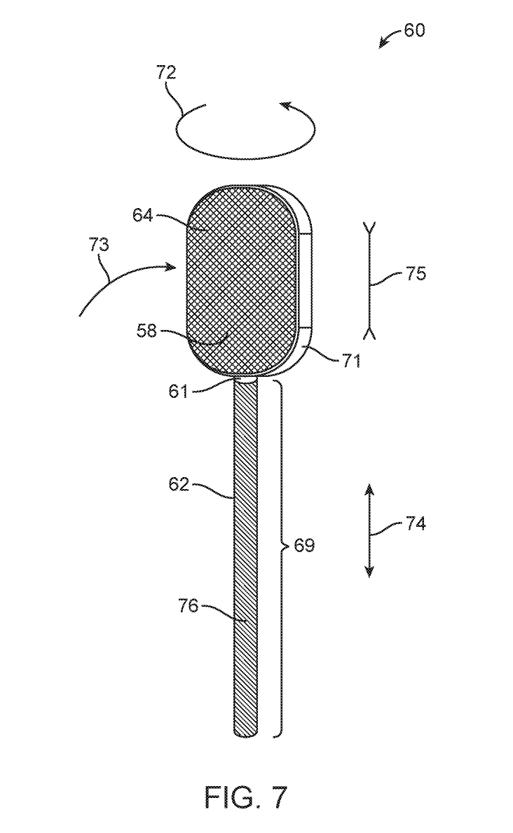

As shown in FIG. 7, the device 60 includes a rigid or semi-rigid plastic handle 62 and an applicator tip 64 that is formed in or attached to the handle 62 according to still another embodiment of the invention.

The handle 62 includes a single compartment containing a reservoir 69 holding a diluent 76. This reservoir 69, contained inside the handle 62, may occupy most, all, or a fraction of the handle. The handle 62 may be of any length, shape or other configuration so as to allow access to the oral mucosa without the caregiver contacting the inside of the patient's mouth.

The reservoir 69 and applicator tip 64 are separated by a partition 61 and, when intact, prevents diluent 76 flow from reservoir 69 to the application layer 58. The partition 61 may be configured in a perpendicular orientation relative to the handle length as shown in FIG. 7. Of course, other configurations, including diagonal, convex, concave, etc., are possible. The partition may be constructed of any breakable non-reactive substance including glass or plastic, for example.

The applicator tip 64 includes a non-reactive, porous, application layer 58, such as a foam that optionally has an abrasive outer surface. A cellophane cover 25 (FIG. 2) covers the outer surface of the application layer 58 (or the entire applicator tip 64). The applicator tip 64 has a single-compartment design and includes a rigid or semi-rigid backing 71 and an application layer 58 that is attached to the backing 71. The application layer 58 is pre-loaded with a suitable amount of a pharmaceutical formulation that includes an effective amount of the active ingredient. The active ingredient may be epinephrine or naloxone.

The partition 61 may be broken by twisting 72, bending 73, pulling 74 or compressing 75 the handle 62 of the device 60 relative to the applicator tip 64, for example. Breaking the partition causes the diluent 69 to flow from the reservoir 69 into the applicator tip 64 and mix with an effective amount of active ingredient contained in the applicator tip 64 of the application layer 58.

Holding the device 60 with the application layer 58 facing down for a few seconds uses gravitational force to help fill the application layer 58 with the diluent 76. The user then removes the cellophane cover and applies the pharmaceutical formulation to the oral mucosa (upper and lower lips and/or buccal region). If the patient is not revived after 2-10 minutes, the user needs to discard the device and administer another dose by repeating the procedure using a second new device 60. This sequence of steps can be repeated as needed.

Single Compartment Configuration Separate from Handle

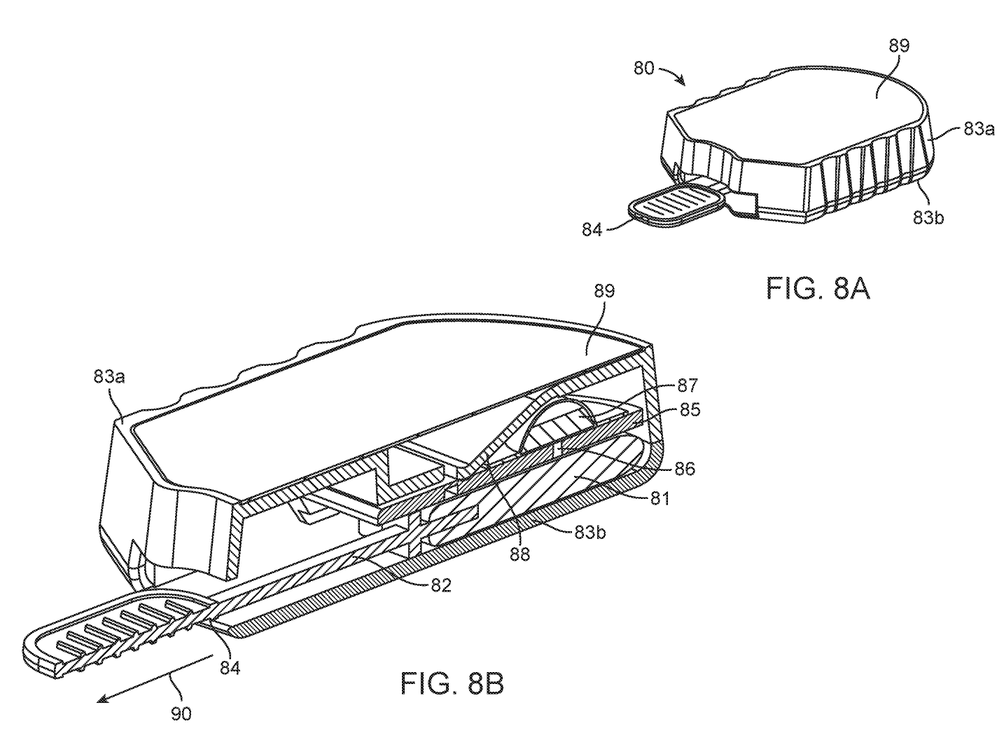

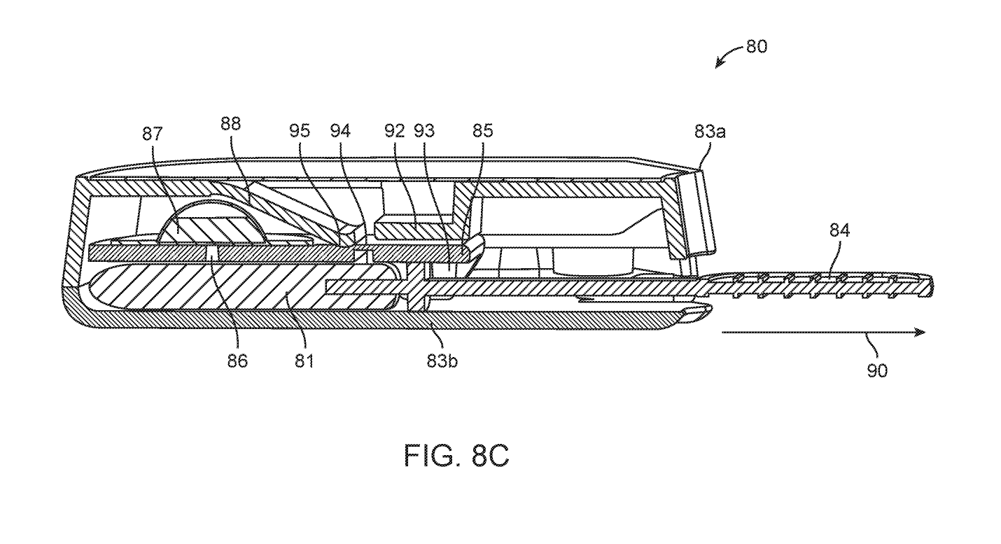

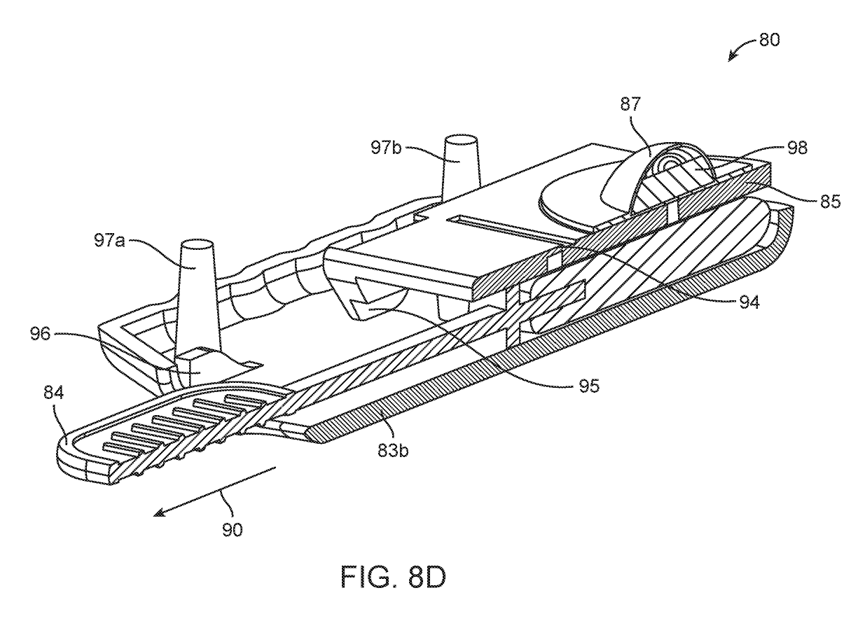

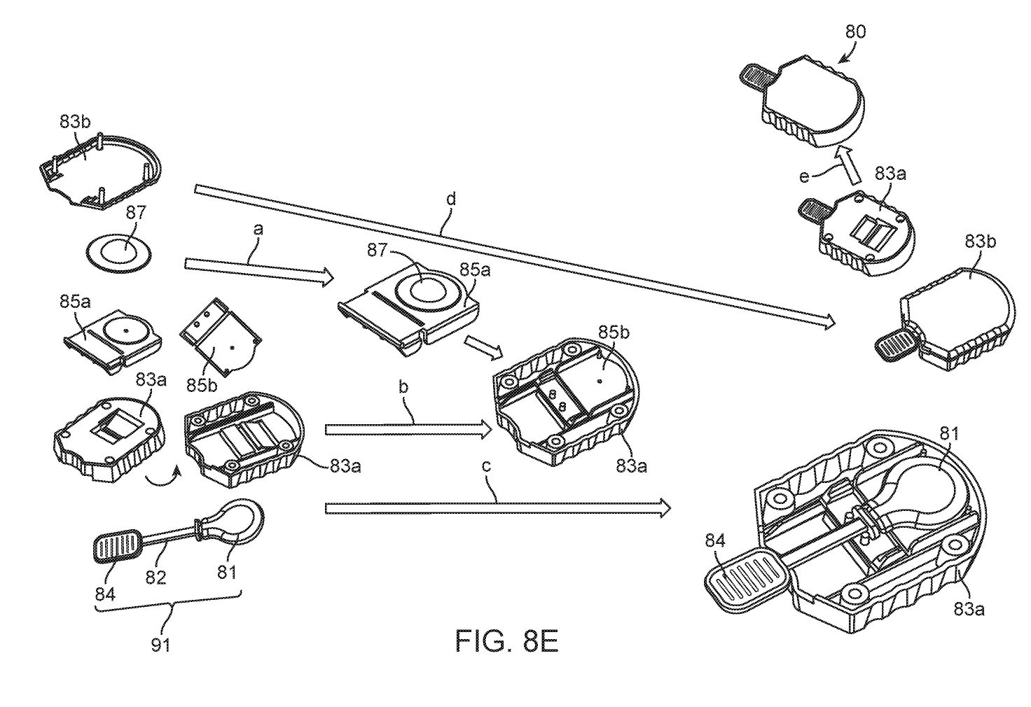

A hand-held device 80 for urgent systemic delivery of a pharmaceutical composition across oral mucosae is shown in FIG. 8A. Instructions for use may be included on a sticker 89 applied to the upper half (i.e., top) of the housing 83a, for example. FIG. 8B is a cross sectional perspective view of the device shown in FIG. 8A including design elements and internal features. The device 80 comprises an applicator tip 81, a handle 82, and a molded housing. The housing may have an upper 83a half and lower 83b half (i.e., base) fastened together post assembly. The applicator tip 81 comprises an exterior surface configured to receive and apply the pharmaceutical composition to the oral mucosae. The elongated rigid or semi-rigid handle 82 has a grasping end 84. End 84 may have a texture or surface that prevents the fingers from slipping when the end 84 is gripped. The applicator tip 81 is positioned on the handle 82 opposite the grasping end 84. The removable housing 83a, 83b is configured to enclose the applicator tip 81 and a portion of the elongate handle 82 excluding the grasping end 84 which protrudes out from an end of the housing 83a, 83b. The removable housing contains a moveable shuttle 85 having a first side 85a, a second side 85b, and at least one opening 86 between the sides 85a, 85b. The shuttle may be moved relative to the stationary housing in a slidable motion, or example. The removable housing also contains an internal reservoir 87 configured to contain an effective amount of the pharmaceutical composition. The composition includes a pharmaceutically active ingredient and a physiologically acceptable carrier. The reservoir 87 is positioned on the first side 85a of the slidable shuttle 85 and positioned in communication with the at least one opening 86. The opening 86 may be a hole or slot, for example. The applicator tip 81 is positioned on the second side 85b of the slidable shuttle and positioned in communication the at least one opening 86. The removable housing also contains a barrier between the reservoir 87 and applicator tip 81 that prevents the pharmaceutical composition from flowing from the reservoir 87 through the at least one opening 86 to the applicator tip 81. Additionally, the removable housing contains a means for moving the applicator tip 81 and the shuttle 85 to disrupt the barrier so as to permit flow of the pharmaceutical composition from the internal reservoir 87 through the at least one opening 86 onto the exterior surface of the applicator tip 81.

The means for moving the applicator tip 81 and the shuttle 85 to disrupt the barrier includes holding the removable housing with the fingers of one hand while pulling the grasping end 84 of the handle 82 away from the housing with fingers on the other hand until the application tip 81 separates (i.e., breaks free) from the housing 83a, 83b. The active ingredient is naloxone or epinephrine. The physiologically acceptable carrier may be an alcohol (e.g., ethanol) and the application layer contains a plant-based resin (e.g., benzoin gum or badam gum). Additional plant-based products, including mastic and mucilage are also contemplated for potential use. Other mucoadhesive polymers including polysaccharides, proteins, polyethers, polyesters, poloxamers, or RP polymers that form a bond between mucus and polymer via physical or chemical bioadhesion, including (but not limited to) electrostatic or hydrophilic interactions, hydrogen bonding or dispersion forces, may be used. The reservoir 87 may be a blister containing between about 100 μL and 500 μL of the pharmaceutical composition. The barrier is disrupted when the reservoir 87 contacts a fixed portion 88 of the housing 83a, 83b when the grasping end 84 is pulled away from the housing in direction 90. Like the grasping end 84, the sides of the housing may include a texture or material that improves the grip to facilitate removal of the application tip 81 from the housing. This removal completely separates the buccal swab 91 from the housing. The fixed portion 88 may be a spring loaded CAM built into the top cover 83a of the housing, for example.

FIG. 8C is a cross section of the devices shown in FIGS. 8A-8B, respectively. A retention detent 95 in the shuttle 85 contacts the fixed portion 88 to hold components secure until use of the device is required. When treatment is required, the user simply pulls the grasping end 84 in direction 90 while holding the housing steady. This slides the shuttle 85 (with reservoir 87) and application tip 81 in direction 90. This movement causes the reservoir 87 to contact a fixed portion 88 (e.g., a spring loaded CAM) which exerts increasing angular pressure on the reservoir 87 causing a controlled squeeze on the reservoir 87 which ultimately ruptures the reservoir 87. This, in turn, causes the releases of the pharmaceutical ingredients 98 (FIG. 8D) from the reservoir 87 to flow through at least one opening 86 and onto the application tip 81. The reservoir 87 is bolstered 92 to prevent reservoir expansion and the fixed portion 88 then pushes the applicator tip retention zone 93 on the shuttle 85 toward the top of the housing 83a to clear additional space for the applicator tip 81, now impregnated with the pharmaceutical composition, to be released and removed completely from the housing confines.

The top half 83a of the housing has been removed from assembly posts 97a, 97b in FIG. 8D to further illustrate how the application tip disengages from the housing. The shuttle 85 with hinge 94, located between the reservoir 87 and the retention zone 93, slides along follower 95 in direction 90 in a sled-like manner until the follower 95 contacts CAM 96. The CAM 96 deflects the application tip retention zone 93 to disengage retention pins. This facilitates the exit of the application tip 81 from the housing in direction 90. The application tip 81 can now be placed in contact with the mucosa of the patient requiring medical attention. This entire process takes only a matter of seconds to complete.

Turning now to FIG. 8E, an exploded view of the parts and assembly relating to this embodiment are shown. Basic components include the molded base of the housing 83b, reservoir 87, shuttle 85 (including top and bottom sides 85a and 85b, respectively), molded top of the housing 83a, and the buccal swab 91. The reservoir 87 may take the form of a storage blister and the shuttle 85 may be made of polypropylene or similar material known to those of skill in the art. The swab 91 includes the applicator tip 81, the elongate handle 82 and the grasping end 84.

Assembly may include: a) installing the reservoir 87 on the first side 85a of the slidable shuttle 85; b) mounting the shuttle 85 on the housing 83a; c) placing the applicator tip 81 of the swab 91 against the second side 85b of the slidable shuttle 85; d) securing the base 83b of the housing against the top 83a of the housing so as to enclose all parts within the interior of the housing except the grasping end 84 and a portion of the handle 82 of the swab 91; and e) optionally rotating the housing and applying an instructional sticker on the exterior of the housing. These steps are for exemplary purposes only. Many other combinations of assembly and manufacture are, of course, possible.

Foam Material and Chemical Treatment

To reduce non-specific adsorption of the drug on the foam surface, it is best to use a hydrophilic foam such as polyurethane that does not react with the pharmaceutical formulation. To further reduce non-specific adsorption, the foam, Velcro® or plastic bristles can be treated, for example, with one of the following solutions: 1) Pluorinic®—block copolymer of polyethylene glycol and polypropylene glycol. Pluronic® F-127 (Sigma Aldrich, St. Louis, Mo.) is typically used for this purpose, but there are several products in this family of products; and 2) Fluorinert™—completely fluorinated hydrocarbon. An example is Fluorinert™ FC-70 (Sigma Aldrich, St. Louis, Mo.).

Drug Product

The drug product can take a number of forms. According to one embodiment of the invention, the drug product is a pharmaceutical composition comprising the active ingredient (e.g., epinephrine or naloxone), resin and a volatile solvent and, optionally, water, such as the composition described in U.S. Patent Application Publication 2014/0371210 A1, the disclosure of which is hereby incorporated by reference in its entirety for all purposes.

### Working Example 1

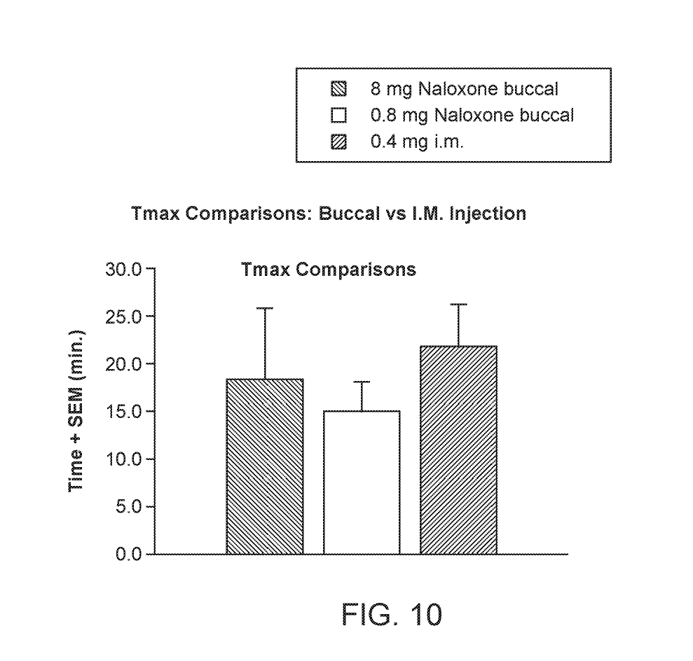

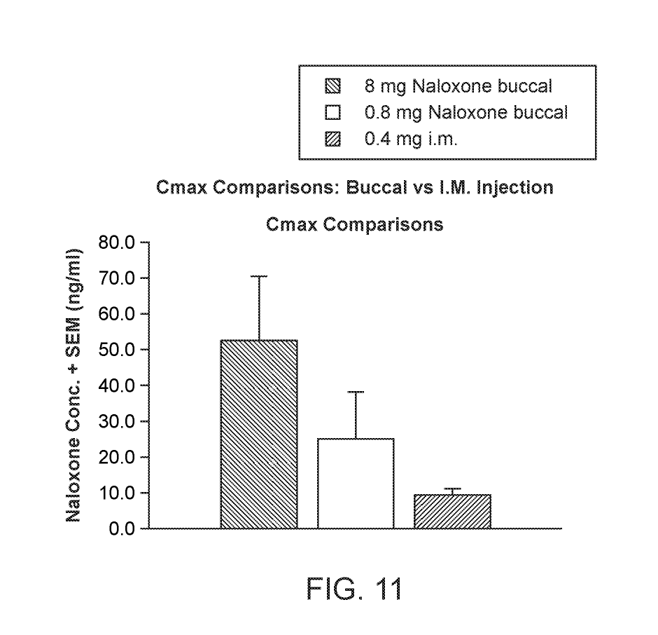

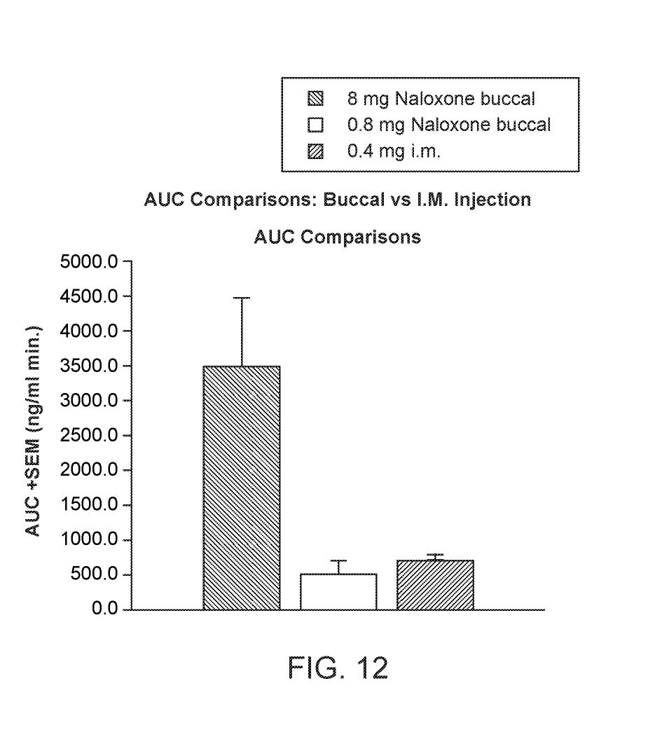

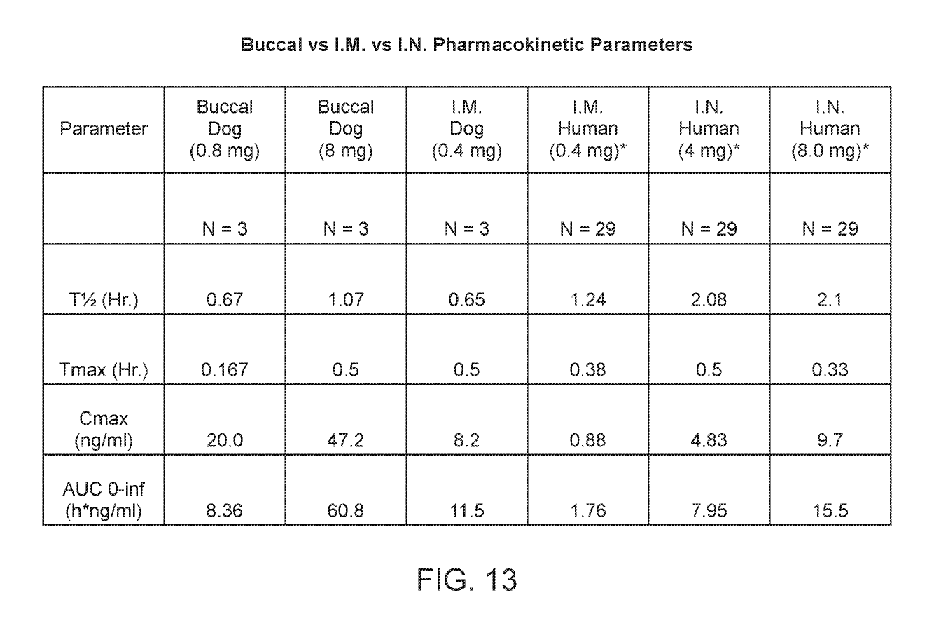

FIGS. 9-13 relate to outcomes from a canine (i.e., dog) PK study of naloxone delivered via buccal route compared to intramuscular administration.

i) 4 mg/ml of naloxone (in benzoin solution) were prepared. 200 microliters were pipetted onto a swab and delivered to the right side of the buccal mucosa of a canine for 10 seconds. The delivered dose was 0.8 mg.

ii) 40 mg/ml of naloxone (in benzoin solution) were prepared. 200 microliters were pipetted onto a swab and placed against the right side of the buccal mucosa of a canine for 10 seconds. The delivered dose was 8 mg.

iii) 0.4 mg intramuscular (IM) commercial injection of naloxone was administered to the canine. The delivered does was 1 ml injection of 0.4 mg/ml.

The study design included a three dog (i.e., Canis familiaris), three way crossover study. Each dog received each of the two buccal solution and an IM injection on successive weeks such that there was a one week rest interval between dosing groups. Blood samples of each canine were taken for PK analysis.

| | | 8.0 mg | 0.8 mg | |
|---|---|---|---|---|
| TEST | FIGURE | BUCCAL | BUCCAL | 0.4 mg INTRA- |
| PARAMETER | NUMBER | SWAB | SWAB | MUSCULAR |
| Tₘₐₓ | 9 | 18 | 15 | 22 min. |
| Cₘₐₓ | 10 | 52 | 25 | 9 ng/ml |
| AUC | 11 | 3500 | 500 | 600 ng/ml-min. |

Buccal delivery of naloxone at the doses tested compared very favorably to the standard intramuscular (IM) dose. The higher Cₘₐₓ and shorter Tₘₐₓ indicates a quicker uptake of the drug from the oral mucosa than from muscle. In addition to the favorable PK profile, buccal route offers a simple, painless and safe alternative to the intramuscular (IM) or intranasal (IN) route particularly for untrained personnel who may be uncomfortable with needles or with the procedures for intranasal delivery. This simple ease of administration could provide broader use of these products enabling family, friends, caregivers and general public to administer naloxone in life threatening situations and increase saving lives. Also, the higher Cₘₐₓ, may be important in the current opioid epidemic as many patients who overdose on drugs such a fentanyl have a higher dose requirement for naloxone reversal due to the higher potency of these newer agents.

In summary, buccal administration of naloxone offers an effective new way for untrained personnel to quickly deliver therapeutic doses of naloxone to a patient who has overdosed on opioids to increase their chance of survival. Similar results can be expected from other regions of the oral mucosa (i.e., sublingual or labial) as well.

### Working Example 2

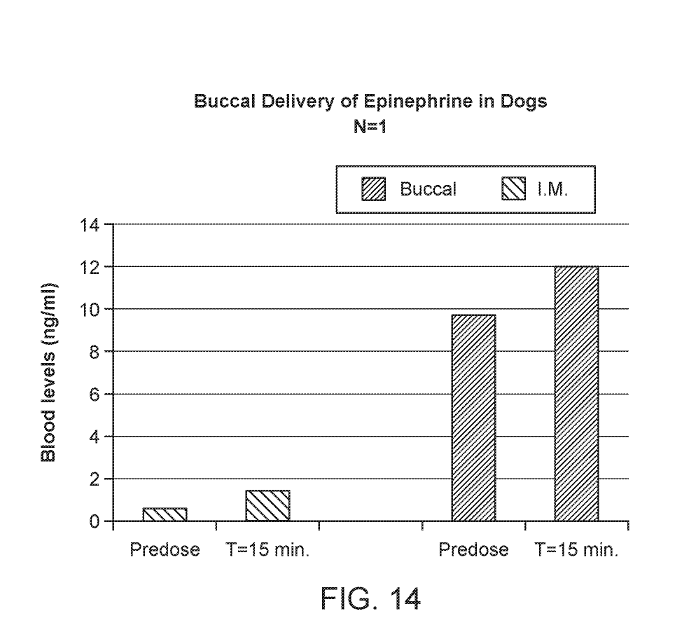

FIG. 14 relates to an outcome from a canine PK study of epinephrine delivered via buccal route compared to intramuscular administration.

The study design included using a Beagle (i.e, Canis familiaris). One dog received a buccal solution swab and one dog received an IM injection. Blood samples of the canine were taken for PK analysis.

| | | 4-5 mg | |
|---|---|---|---|
| TEST | FIGURE | BUCCAL | 0.3 mg |
| PARAMETER | NUMBER | SWAB | INTRAMUSCULAR |
| Tₘₐₓ | 13 | 12 ng/ml | 1.7 ng/ml |

FIG. 14 is a histogram of Tₘₐₓ comparisons of buccal versus intramuscular injection of epinephrine according to some embodiments of the invention. The epinephrine data is from two separate experiments using a dog study model. Epinephrine is an endogenous catecholamine hormone with levels that can fluctuate rapidly due to many factors, including the excitability of the dogs being prepared for a PK study, for example. Additionally the half-life of epinephrine is between about 3-8 minutes. This explains variability in basal levels that fluctuate rapidly (e.g., within seconds). Dog plasma levels were analyzed via MS/LC.

FIG. 14 and the table above shows the blood levels of epinephrine administered intramuscularly and buccally. The epinephrine can be delivered via the buccal route of administration, over and above the basal level, using a plant-based resin (tincture of benzoin, 75-80% ETOH) applied by a flocked swab and was approximately 20% of the increase demonstrated by benchmark intramuscular injection. The dog injected intramuscularly received 0.3 mg epinephrine while the dog receiving the buccal swab received approximately 4-5 mg. epinephrine. Fifteen minutes equaled the Tₘₐₓ.

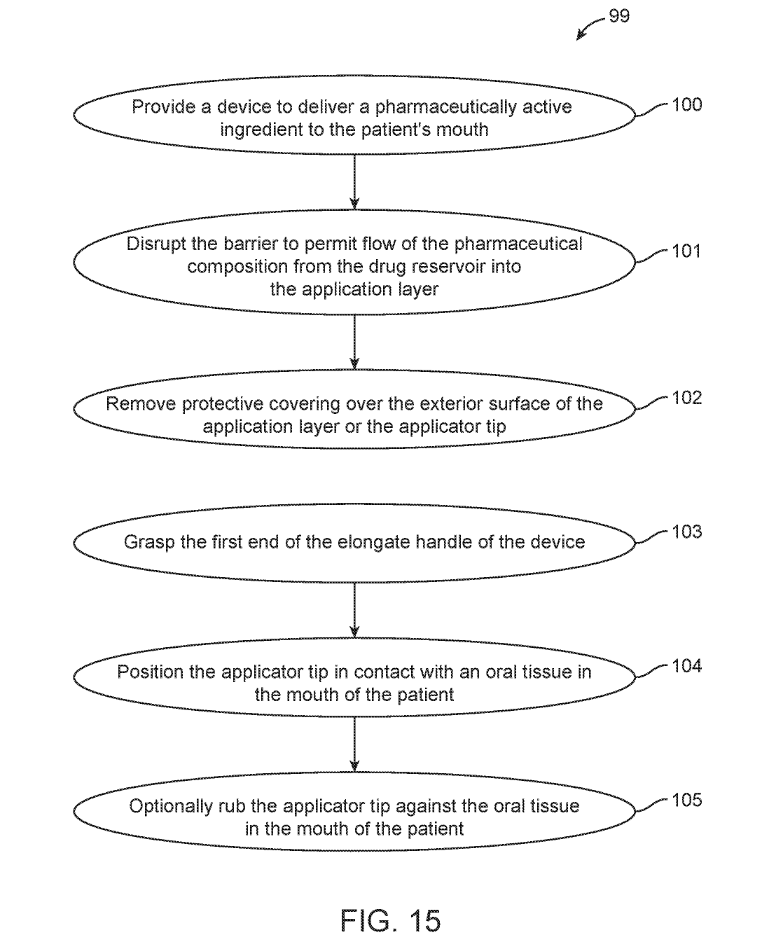

FIG. 15 shows a flowchart illustrating another method 99 of using the device to deliver a pharmaceutically active ingredient according to other embodiments of the invention. A device containing a pharmaceutically active ingredient is first provided 100. A barrier is disrupted to permit flow of the pharmaceutical composition from the drug reservoir into the application layer 101. The protective covering is removed 102. The first end of the elongate handle is grasped 103. The applicator tip is positioned substantially in contact with an oral tissue in the mouth of the patient 104. Optionally, the applicator tip can be rubbed against the oral tissue of the patient 105.

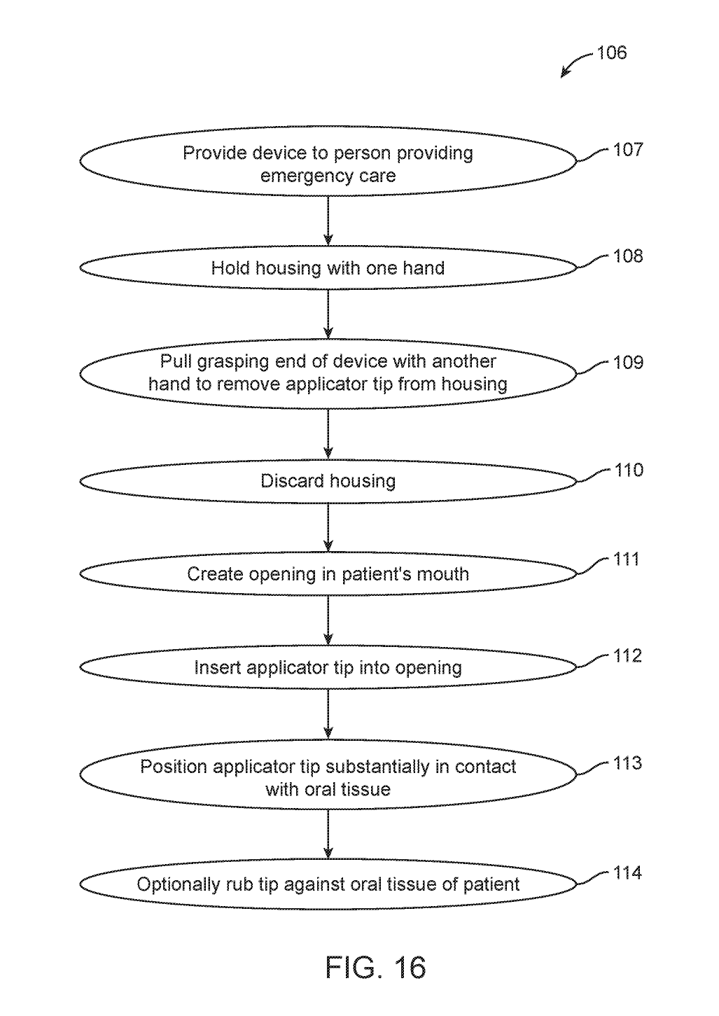

FIG. 16 is a flowchart illustrating a method 106 of using the device to deliver a pharmaceutically active ingredient to a mouth of a patient requiring emergency medical care from a personal provider. The method 106 includes first providing the person with a device 107. The housing is held with a first hand of the person 108. The person uses their second hand to pull the grasping end of the device to remove the applicator tip of the device from the housing 109. Pulling the applicator tip causes the reservoir to break and flow the pharmaceutically active ingredient onto the applicator tip as the tip is being pulled apart from the housing. The housing can now be discarded 110. An opening is created 111 (by the person) to insert the application tip in the mouth of the patient by pulling the patient's cheek away from their mouth. The applicator tip is inserted into the opening 112 and substantially positioned in contact with an oral tissue of the patient 113. Optionally, the applicator tip can be rubbed against the oral tissue of the patient 114. This may facilitate absorption of the pharmaceutically active ingredient through the mucosa and to expedite system delivery.

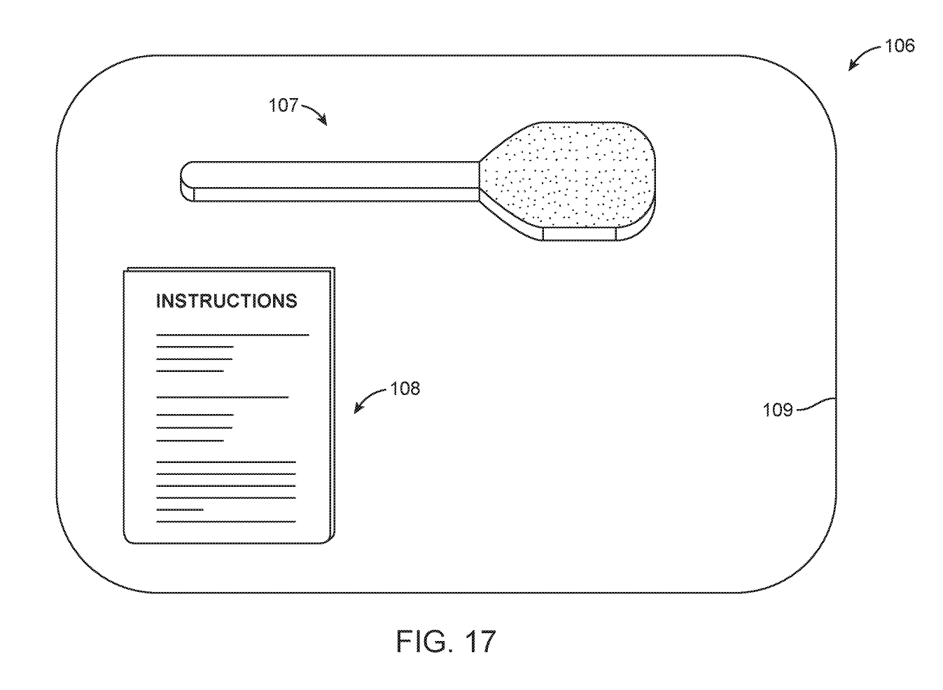

FIG. 17 depicts a kit 106 for the urgent delivery of a pharmaceutically active ingredient to a mucosa of a patient designed to be easily administered during a medical emergency. The kit 106 includes a device 107 according to any of the aforementioned embodiments. It also includes instructions 108 for using the device properly. The instructions 108 may be in visual, audio, electronic or other formats. They may also take for form of a sticker applied to the device 106 itself. A protective shield may be placed over the applicator tip or the entire device and hermetically sealed. A package 109 containing the serializable device and the instructions for use is also included.

Although embodiments of the invention have been described in considerable detail with reference to certain preferred versions thereof, other embodiments are possible.

Therefore, the spirit and scope of the appended claims should not be limited to the descriptions of the embodiments above.

What is claimed is:

1. A hand-held device for delivery of a pharmaceutically active ingredient, the device comprising:
(a) an elongated rigid or semi-rigid handle having an end;
(b) an applicator tip comprising:
(i) a drug reservoir configured to contain a pharmaceutical composition comprising an effective amount of the active ingredient, wherein the drug reservoir comprises an exterior surface that is formed in or attached to the end of the handle, and an open end;
(ii) a porous application layer comprising an exterior surface configured to spread the pharmaceutical composition at a delivery site and an interior surface;
(iii) a barrier between the open end of the drug reservoir and the interior surface of the application layer and configured to prevent flow of the pharmaceutical composition from the drug reservoir to the application layer; and wherein the barrier is further configured to rupture when sufficient pressure is applied to the barrier to permit flow of the pharmaceutical composition from the drug reservoir into the application layer, and
(c) a removable protective covering over the exterior surface of the application layer or the applicator tip.

2. The device of claim 1 wherein the application layer comprises an absorbent foam.

3. The device of claim 1 wherein the exterior surface of the application layer is configured to abrade oral tissue at the delivery site.

4. The device of claim 3 wherein the oral tissue is oral mucosa and the delivery site is buccal, sublingual or labial.

5. The device of claim 4 wherein the oral mucosa is buccal mucosa.

6. The device of claim 1 wherein the pharmaceutical composition comprises a unit dose of the active ingredient.

7. The device of claim 1 wherein the active ingredient is epinephrine or naloxone.

8. The device of claim 1 wherein the pharmaceutical composition comprises the pharmaceutically active ingredient, a resin, a volatile solvent, and optionally water.

9. The device of claim 1 wherein the barrier comprises a tab that is configured to displace or disrupt the barrier when the tab is pulled.

10. The device of claim 1 wherein the barrier comprises a button attached to the interior surface of the application layer, the button comprising one or more spiky projections that extend toward the nonporous barrier, wherein the button is configured to pierce the barrier to permit flow of the pharmaceutical composition from the drug reservoir to the application layer when pressure is exerted against the exterior surface of the application layer.

11. A device for delivery of a pharmaceutically active ingredient, the device comprising:
(a) an elongated rigid or semi-rigid handle having an end;
(b) an applicator tip comprising:
(i) a rigid, non-porous backing comprising an exterior surface that is formed in or attached to the end of the handle and an interior surface; and
(ii) a porous application layer comprising a first surface in contact with the interior surface of the backing and a second surface configured to spread the pharmaceutical composition on oral mucosa of a patient, wherein the application layer contains a pharmaceutical composition comprising an effective amount of the active ingredient; and
(c) a removable protective covering surrounding the applicator tip.

12. The device of claim 11 wherein the application layer comprises an absorbent foam, a hook-and-eye fastener material, or nylon bristles.

13. The device of claim 11 wherein the exterior surface of the application layer is configured to abrade the oral mucosa at a drug delivery site.

14. The device of claim 11 wherein the pharmaceutical composition comprises a unit dose of the active ingredient.

15. The device of claim 11 wherein the active ingredient is an adrenergic hormone or a derivative of morphine.

16. The device of claim 11 wherein the active ingredient is epinephrine or naloxone.

17. The device of claim 11 wherein the pharmaceutical composition comprises the pharmaceutically active ingredient, a resin, a volatile solvent, and optionally water.

18. A method of delivering a pharmaceutically active ingredient to a mouth of a patient requiring emergency medical care, the method comprising:
providing a device having an elongated handle at a first end and an applicator tip at a second end, the applicator tip containing a pharmaceutically active ingredient;
grasping the first end of the elongate handle of the device; and
positioning the applicator tip substantially in contact with an oral tissue in the mouth of the patient.

19. The method of claim 18 wherein an exterior surface of the application layer is configured to abrade the oral tissue; and further comprising:
optionally rubbing the applicator tip against the oral tissue in the mouth of the patient.

20. The method of claim 18 wherein the oral tissue includes mucosae selected from the group consisting of buccal mucosa, sublingual mucosa and labial mucosa.

21. The method of claim 20 wherein the oral tissue is buccal mucosa.

22. A hand-held device for urgent systemic delivery of a pharmaceutically active ingredient and a physiologically acceptable carrier across oral mucosae, the device comprising:
(a) an applicator tip comprising:
(i) a porous application layer comprising an exterior surface configured to receive and apply the pharmaceutically active ingredient to the oral mucosa;
(b) an elongated rigid or semi-rigid handle having an end, the handle comprising:
(i) a first internal reservoir configured to contain an effective amount of the active ingredient; and
(ii) a second internal reservoir configured to contain the physiologically acceptable carrier;
(iii) a barrier between the first and second reservoirs configured to prevent mixing of the active ingredient with the physiologically acceptable carrier; and
(iv) a means for moving or disrupting the barrier to permit mixing of the active ingredient and the physiologically acceptable carrier, wherein the mixture of the active ingredient and the carrier flows from the handle into the application layer of the application tip, and
(c) a removable protective covering over the exterior surface of the application layer or the applicator tip.

23. The device of claim 22 wherein the active ingredient is naloxone or epinephrine.

24. The device of claim 22 wherein the physiologically acceptable carrier is a diluent and the diluent is ethanol.

25. The device of claim 22 wherein the means for moving or disrupting the barrier comprises a partition that is configured to displace or disrupt the barrier when the handle is twisted and/or bent.

26. The device of claim 22 wherein the application layer contains a plant-based resinous gum configured to provide mucoadhesive properties.

27. The device of claim 22 wherein the oral mucosae is buccal mucosa.

28. A hand-held device for urgent systemic delivery of a pharmaceutical composition across oral mucosae, the device comprising:
(a) an applicator tip comprising a porous application layer comprising an exterior surface configured to receive and apply the pharmaceutical composition to the oral mucosae;
(b) an elongated rigid or semi-rigid handle having an end, the handle comprising:
(i) an internal reservoir configured to contain an effective amount of the pharmaceutical composition, the composition including a pharmaceutically active ingredient and a physiologically acceptable carrier;
(ii) a barrier between the reservoir and applicator layer configured to prevent the pharmaceutical composition from flowing to the application layer of the application tip; and
(iii) a means for moving or disrupting the barrier to permit flow of the pharmaceutical composition from the internal reservoir in the handle to the application layer of the application tip.

29. The device of claim 28 wherein the active ingredient is naloxone or epinephrine.

30. The device of claim 28 wherein the physiologically acceptable carrier is ethanol.

31. The device of claim 28 wherein the application layer contains a plant-based resin.

32. The device of claim 31 wherein the plant-based resin is benzoin gum or badam gum.

33. The device of claim 28 further comprising a removable protective covering over the exterior surface of the application layer or the applicator tip.

34. The device of claim 28 wherein the barrier between the reservoir and applicator layer is a foil blister, the applicator tip is a flocked pad material and the means for moving or disrupting the barrier to permit flow of the pharmaceutical composition from the internal reservoir in the handle to the application layer of the application tip includes breaking the foil blister.

35. A hand-held device for urgent systemic delivery of a pharmaceutical composition across oral mucosae, the device comprising:
(a) an applicator tip comprising an exterior surface configured to receive and apply the pharmaceutical composition to the oral mucosae;
(b) an elongated rigid or semi-rigid handle having a grasping end; wherein the applicator tip is positioned on the handle opposite the grasping end;
(c) a removable housing configured to enclose the applicator tip and a portion of the elongate handle excluding the grasping end; the removable housing containing:
(i) a slidable shuttle having a first side, a second side, and at least one opening between the sides;
(ii) an internal reservoir configured to contain an effective amount of the pharmaceutical composition, the composition including a pharmaceutically active ingredient and a physiologically acceptable carrier; wherein the reservoir is positioned on the first side of the slidable shuttle and positioned in communication with the at least one opening, wherein the applicator tip is positioned on the second side of the slidable shuttle and positioned in communication the at least one opening;
(iii) a barrier between the reservoir and applicator tip configured to prevent the pharmaceutical composition from flowing from the reservoir through the at least one opening to the applicator tip; and
(iv) a means for moving the applicator tip and the shuttle to disrupt the barrier so as to permit flow of the pharmaceutical composition from the internal reservoir through the at least one opening onto the exterior surface of the applicator tip.

36. The device of claim 35 wherein the means for moving the applicator tip and the shuttle to disrupt the barrier includes holding the removable housing while pulling the grasping end of the handle away from the housing until the application tip is separated from the housing.

37. The device of claim 35 wherein the active ingredient is naloxone or epinephrine.

38. The device of claim 35 wherein the physiologically acceptable carrier is ethanol.

39. The device of claim 35 wherein the application layer contains a plant-based resin.

40. The device of claim 39 wherein the plant-based resin is benzoin gum or badam gum.

41. The device of claim 35 wherein the reservoir is a blister, wherein the blister contains between about 100 μL and 500 μL of the pharmaceutical composition.

42. The device of claim 25 wherein the barrier is disrupted when the reservoir contacts a fixed portion of the housing.

43. A hand-held device to expedite delivery of a pharmaceutically active ingredient to an oral cavity of a patient, the device comprising:
(a) an elongated rigid or semi-rigid handle having an end, the handle comprising:
(i) a first reservoir configured to contain a diluent, wherein the first reservoir comprises an exterior surface that is formed in or attached to the end of the handle, and an open end;
(b) an applicator tip comprising:
(i) a second reservoir configured to contain an effective amount of the active ingredient, wherein the second reservoir comprises an exterior surface that is attached to the end of the handle, and an open end;
(ii) a porous application layer comprising an exterior surface configured to receive and spread the diluent and active ingredient to the oral cavity of the patient when mixed together;
(iii) a barrier between the first and second reservoirs configured to prevent mixing of the diluent and the active ingredient; and
(iv) a means for moving or disrupting the barrier to permit flow of the diluent to the applicator tip and mixing with the active ingredient in the application layer, and
(c) a removable protective covering over the exterior surface of the application layer or the applicator tip.

44. The device of claim 43 wherein the means for moving or disrupting the barrier comprises moving the applicator tip in a direction while the handle remains substantially stationary to displace or disrupt the barrier when movement is applied.

45. The device of claim 43 wherein moving the applicator tip includes twisting, bending, pulling, compressing, or any combination thereof.

46. The device of claim 43 wherein the application layer is configured to spread the diluent and active ingredient substantially across buccal mucosa.

47. A method of delivering a pharmaceutically active ingredient to a mouth of a patient requiring emergency medical care from a person, the method comprising:
providing the person with a device having an elongated handle positioned between a grasping end and an applicator tip, the applicator tip configured to be removably contained within a housing; the housing containing a reservoir filled with the pharmaceutically active ingredient and a mechanism for breaking the reservoir;
holding the housing with a first hand of the person;
pulling the grasping end of the device with a second hand of the person so as to remove the applicator tip of the device from the housing; wherein pulling the applicator tip causes the reservoir to break and flow the pharmaceutically active ingredient onto the applicator tip as the tip is being pulled apart from the housing;
discarding the housing;
creating an opening to insert the device in the mouth of the patient by pulling a cheek away from the mouth of the patient;
inserting the applicator tip into the opening;
positioning the applicator tip substantially in contact with an oral tissue of the patient; and
optionally rubbing the applicator tip against the oral tissue of the patient.

48. A method of manufacturing a hand-held device for urgent delivery of a pharmaceutically active ingredient to a mucosa of a patient, the method comprising:
providing and molding a medical grade material for manufacturing an elongated rigid or semi-rigid handle having an end; and
providing and molding a medical grade material for manufacturing an applicator tip, wherein the applicator tip comprises:
(i) a drug reservoir configured to contain a pharmaceutical composition comprising an effective amount of the active ingredient, wherein the drug reservoir comprises an exterior surface that is formed in or attached to the end of the handle, and an open end;
(ii) a porous application layer comprising an exterior surface configured to spread the pharmaceutical composition at a delivery site and an interior surface;
(iii) a barrier between the open end of the drug reservoir and the interior surface of the application layer and configured to prevent flow of the pharmaceutical composition from the drug reservoir to the application layer; and
(iv) a means for moving or disrupting the barrier to permit flow of the pharmaceutical composition from the drug reservoir into the application layer, and a removable protective covering over the exterior surface of the application layer or the applicator tip;
placing and sealing the pharmaceutical composition in the drug reservoir; and
assembling and packaging the hand-held device.

49. A method of treating an individual comprising:
providing a device comprising:
(i) an elongated rigid or semi-rigid handle having an end;
(ii) an applicator tip formed in or attached to the end of the handle, the applicator tip comprising:
a drug reservoir configured to contain a pharmaceutical composition comprising an effective amount of naloxone or epinephrine, wherein the drug reservoir comprises an exterior surface that is formed in or attached to the end of the handle, and an open end; and
an exterior surface in fluid communication with the drug reservoir that is configured to spread the pharmaceutical composition on an oral mucosa of the individual; and
(iii) a removable protective covering over the exterior surface of the application layer or the applicator tip;
removing the protective covering;
inserting the tip into the mouth of the individual; and
spreading the pharmaceutical composition on the oral mucosa.

50. The method of claim 49, wherein the individual is unconscious or unresponsive.

51. A kit for the urgent delivery of a pharmaceutically active ingredient to a mucosa of a patient during a medical emergency, the kit comprising:
a device of any of the preceding claims;
instructions for using the device; and
a package containing the device and the instructions for use.
